(19) United States
(12) Patent Application Publication
(10) Pub. No.: US 2021/0115177 A1
(43) Pub. Date: Apr. 22, 2021

(54) AMPHIPHILIC POLYMERS, PROCESS OF PREPARING SAME AND USES THEREOF

(71) Applicant: TECHNION RESEARCH & DEVELOPMENT FOUNDATION LTD., Haifa (IL)

(72) Inventor: Alejandro Dario SOSNIK, Haifa (IL)

(21) Appl. No.: 17/251,802

(22) PCT Filed: Jun. 13, 2019
(86) PCT No.: PCT/IL2019/050672

§ 371 Date: Dec. 13, 2020

Related U.S. Application Data
(60) Provisional application No. 62/684,829, filed on Jun. 14, 2018.

(51) Int. Cl. C08F 251/00; A61K 9/107; A61K 47/32; A61K 47/34; A61K 47/24; A61K 45/06; A61K 47/36; A61K 31/506

(52) U.S. Cl. CPC C08F 251/00; A61K 9/1075; A61K 47/32; A61K 31/506; A61K 47/24; A61K 45/06; A61K 47/36; A61K 47/34

(57) ABSTRACT

Compositions comprised of an amphiphilic block polymer, configured to form multi-micellar structures having hydrophilic and hydrophobic domains, and uses same for encapsulating therein active agents such as hydrophobic therapeutic agents, are disclosed. The disclosed compositions are useful e.g., for orally delivering the active agent encapsulated therein and may further be used for controllably releasing the agents in the physiological environment.

### CROSS REFERENCE TO RELATED APPLICATIONS

This application claims the benefit of priority of U.S. Provisional Patent Application No. 62/684,829 filed on Jun. 14, 2018, entitled “AMPHIPHILIC POLYMERS FOR TARGETING CARBOHYDRATE CELL RECEPTORS OR TRANSPORTERS, PROCESS OF PREPARING SAME AND USES THEREOF”, the contents of which are incorporated herein by reference in their entirety.

### FIELD OF INVENTION

This invention is inter alia directed to amphiphilic polymeric compositions and uses thereof, such as for encapsulating and releasing therapeutically active agents.

### BACKGROUND OF THE INVENTION

“Drug release” refers to the process in which drug solutes migrate from its position within the release-modifying compound or compounds into the medium. Drug release is an important topic in the field of drug delivery for decades. With advancement in material design and engineering, novel materials with increasing complexity and functions have been introduced into the development of drug delivery devices and systems.

Molecules, macromolecules and polymers are vastly used to control drug release. Controlling drug release has direct impact on the biological efficacy, the clinical effect and often times on the quality of life of the target patient population. Factors that influence drug release may include solute diffusion and polymeric matrix swelling.

One of the common features of almost all cancers and also potentially one of their common weaknesses is the increased glucose uptake and increased dependence on glucose as a source of building blocks for cell growth and proliferation, a source for energy, or both.

Nano- and micro-particles hold promise for controlled and targeted drug release and delivery. An ideal drug carrier should not exert harmful effects on normal cells. It should also satisfy requirements of stability, in vivo biocompatibility, and ability of targeted on-demand release.

U.S. Pat. No. 5,173,322 teaches the production of reformed casein micelles and the use of such micelles as a complete or partial replacement of fat in food product formulations.

WO 2003/105607 discloses nano-sized self-assembled structured concentrates and their use as carriers of active materials, particularly lipophilic compounds suitable for pharmaceutical or cosmetic applications or as a food additive.

WO 2001/087227 discloses polymeric micelles which are pH and/or temperature sensitive, and which are used to increase potency of therapeutic agents.

### SUMMARY OF THE INVENTION

The present invention is directed to amphiphilic polymeric compositions and uses thereof, such as for encapsulating and releasing therapeutically active agents.

The present invention is based, in part, on the demonstration of the capability of the self-assembled particles disclosed herein to bind cell receptors or transporters, thereby increase their accumulation in the target cell or cell structure (e.g., nucleus). Further, reduction of the accumulation in off-target cells or cell structures increases efficacy and reduces toxicity of active ingredient encapsulated within the particles.

In one aspect of the invention, there is provided a composition-of-matter comprising one or more amphiphilic copolymers, the one or more amphiphilic copolymers comprises a hydrophobic domain and a hydrophilic domain, wherein:
(a) each of the hydrophobic domain and the hydrophilic domain comprises a polymeric backbone having at least two monomeric units;
(b) the polymeric backbone of the hydrophobic domain is attached to the polymeric backbone of the hydrophilic domain; and
(c) the one or more amphiphilic copolymers are configured to form a multi-micellar structure, at a critical micellar concentration (CMC) of below than 4% w/v.

In one embodiment, at least a portion of the hydrophilic domain is capable of binding to a cell or a cell organelle via a carbohydrate cell receptor and/or transporter expressed on the outer surface of the cell or a cell organelle.

In one embodiment, the composition-of-matter is in the form of multi-micellar structure, wherein the multi-micellar structure comprises a plurality of micelles having a size ranging from 1 nm to 10 μm.

In one embodiment, the hydrophobic domain comprises one or more monomeric units derived from a polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester.

In one embodiment, the hydrophobic domain comprises one or more monomeric units derived from a lipidic molecule, a lipid or phospholipid selected from the group consisting of: fatty acid, fatty alcohol, or a combination thereof.

In one embodiment, the hydrophobic domain comprises one or more monomeric units derived from a polymer selected from the group consisting of: N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, valeronolactone.

In one embodiment, the hydrophilic domain comprises one or more monomeric units derived from a polymer selected from the group consisting of: alginate, galactomannan, hydrolyzed galactomannan, glucomannan, hydrolyzed glucomannan, guar gum, xanthan gum, pectin, hyaluronic acid, chondroitin sulfate, dermatan sulfate, keratin sulfate, levan heparin sulfate, beta-glucan, fucoidan, mannan, fucomannan, galactofucan, fucan, or any combination thereof.

In one embodiment, the hydrophobic domain is present in a concentration of 2% to 90%, by weight, of the amphiphilic block polymer.

In one embodiment, the amphiphilic copolymers are characterized as being substantially non-biodegradable for a period of at least 24 hours in a physiologic environment.

In one embodiment, the amphiphilic copolymers are characterized as being biodegradable in a physiologic environment.

In one embodiment, the amphiphilic copolymers are characterized by a hydrophilic-lipophilic balance (HLB) value that ranges from 1 to 24.

In one embodiment, the composition-of-matter comprises one or more active agents, each of the active agents is independently encapsulated within or attached to the hydrophobic domain.

In one embodiment, one or more active agents are characterized as being stably encapsulated within or attached to the hydrophobic domain in a physiological environment for at least 24 h.

In one embodiment, one or more active agents are selected from the group consisting of a pharmaceutically active agent, a labeling agent, a diagnostic agent, a prophylactic agent, a surface-modifying agent, a tumor-targeting-ligand or moiety.

In one embodiment, one or more active agents are water-insoluble agents.

In one embodiment, at least a portion of the hydrophilic domain of the multi-micellar structure is positively or negatively charged.

In one embodiment, the composition-of-matter is for use in delivery of an active agent to a target cell. In one embodiment, the composition-of-matter is for use in drug delivery.

In another aspect, there is provided a pharmaceutical composition, comprising the composition-of-matter of the invention and a pharmaceutically acceptable carrier.

In one embodiment, the pharmaceutical composition is packaged in a packaging material and identified in print, in or on the packaging material, for use in the treatment of a medical condition treatable by the pharmaceutically active agent.

In one embodiment, the pharmaceutical composition is for use in monitoring or treating cancer.

In another aspect, there is provided a method for treating a medical condition, comprising administering the pharmaceutical composition of the invention to a subject in a need thereof, thereby treating the medical condition.

In one embodiment, the pharmaceutical composition of the invention is administered orally, nasally, ocularly or by inhalation.

In another aspect, there is provided a process of preparing an amphiphilic copolymer being in the form of a self-assembled or a multi-micellar structure, the self-assembled structure further comprising one or more active agents, the process comprising the steps of:
grafting a hydrophobic polymeric backbone to a hydrophilic polymeric backbone, thereby forming an amphiphilic copolymer configured to form the self-assembled or a multi-micellar structure;
mixing the amphiphilic block copolymer with a solvent at a concentration above a predefined minimal concentration thereby forming a dispersion;
adding one or more active agents to the dispersion, thereby forming an amphiphilic polymer being in the form of the self-assembled or multi-micellar structure having attached thereto one or more active agents.

In one embodiment, predefined concentration is a critical micellar concentration (CMC).

In one embodiment, the process further comprising a step of heating the amphiphilic copolymer in an aqueous medium to a temperature ranging from about 30° C. to about 50° C., prior to the step of adding the one or more active agents to the dispersion.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description together with the drawings makes apparent to those skilled in the art how embodiments of the invention may be practiced.

In the drawings:

FIG. 1 presents ¹H-Nuclear magnetic resonance (NMR) spectra of pure galactomannan (GM), pure methyl methacrylate (MMA) and four GM-g-PMMA polymers with growing poly(methyl methacrylate) (PMMA) content.

FIGS. 2A-D present dynamic light scattering (DLS) measurements for the determination of the critical micellar concentration (CMC) of two GM-g-PMMA polymers synthesized with GM:MMA feed weight ratios of 2:1 (FIG. 2A at 37° C., FIG. 2B at 25° C.) and 8:1 (FIG. 2C at 37° C., FIG. 2D at 25° C.).

FIG. 3 presents ¹H-NMR spectra of pure hydrolysed GM (hGM), and four hGM-g-PMMA polymers with growing PMMA content.

FIG. 4 presents Fourier-transform infrared spectroscopy (FTIR) spectra of pure hGM, pure MMA and four hGM-g-PMMA polymers with growing PMMA.

FIG. 5 presents the ¹H-NMR calibration curve using hGM:MMA physical mixtures in DMSO-d6 to determine the PMMA content in the different hGM-g-PMMA polymers.

FIGS. 6A-B present dynamic light scattering (DLS) measurements for the determination of the critical micellar concentration of two hGM-g-PMMA at 25° C.:hGM-PMMA2.3 (FIG. 6A) and hGM-PMMA28 (FIG. 6B). The numbers represent the total PMMA content, by weight.

FIGS. 7A-B presents viability percent of Rh30 and RAW 264.7 cells after incubation with different concentrations of hGM-PMMA28 using the MTT method.

FIGS. 8A-C present confocal microscopy micrographs of Rh30 cells incubated with fluorescently labeled 0.1% w/v hGM-PMMA28 particles (green fluorescence): 4° C. for 1 h (FIG. 8A), 37° C. for 1 h (FIG. 8B) and 37° C. for 4 h (FIG. 8C). Red fluorescence (phalloidin) is for actin and blue fluorescence (DAPI) is for nuclei.

FIGS. 9A-C present imagining flow cytometry of Rh30 cells incubated with fluorescently labeled 0.1% w/v hGM-PMMA28 particles (green fluorescence): 4° C. for 4 h (FIG. 9A), 37° C. for 4 h (FIG. 9B) and 37° C. for 24 h (FIG. 9C).

FIG. 10 presents high-resolution scanning electron microscopy micrographs of drug-free hGM-PMMA particles.

FIG. 11 presents a thermal characterization of the Ga1M-g-PMMA copolymer by Differential Scanning calorimetry (DSC).

FIG. 12 presents a reaction scheme of Ce(IV) initiated graft polymerization.

FIG. 13 presents a graph showing biodistribution of galactomannan polymeric nanoparticles in patient-derived sarcomas as a function of extracellular hGLUT-1 expression.

FIGS. 14A-C present confocal microscopy images showing uptake of fluorescently-labeled 0.1% w/v hGM-PMMA28 particles after incubation with RAW 264.7 cells at 37° C. FIG. 14A presents confocal microscopy image showing distribution of hGM-PMMA28 (green fluorescence) within the cell, nuclear staining (blue fluorescence), actin staining (red fluorescence) and a superimposed image showing a colocalization of the green and red fluorescence. FIG. 14B shows uptake at different time intervals (1 h, 4 h, 24 h). After 1 h incubation, a moderate cellular internalization was detected. At 24 h a significant uptake was observed. FIG. 14C represents a bar graph, showing a relative uptake after 1 h, 4 h and 24 h.

FIGS. 15A-C present confocal microscopy images showing uptake of fluorescently-labeled 0.1% w/v hGM-PMMA28 particles after 24 h incubation with RAW 264.7 cells at 4° C. and at 37° C. FIG. 15A shows an uptake at 4° C. and at 37° C. At 4° C. only a negligible cellular internalization was detected. At 37° C. a significant uptake was observed. FIG. 15B represents a bar graph, showing a relative uptake at 4° C. and at 37° C. FIG. 15C presents confocal microscopy image showing distribution of hGM-PMMA28 (green fluorescence) within the cell, Nuclear staining (blue fluorescence), actin staining (red fluorescence) and a superimposed image showing a colocalization of the green and red fluorescence, after incubation at 4° C. and at 37° C.

FIG. 16 shows a Cryo-TEM image of hGM-PMMA28 particles. Arrows indicate the nanoparticles.

### DETAILED DESCRIPTION OF THE INVENTION

The present invention, in some embodiments thereof, relates to compositions comprising active agents, and methods for treating medical conditions by administering the compositions to a subject in need thereof. The present invention, in further embodiments thereof, relates to compositions and methods for treating medical conditions that are otherwise treatable by parenteral administration of hydrophobic therapeutically active agents and more specifically, but not exclusively, to compositions for oral administration of such therapeutically active agents and to uses thereof in the treatment of medical conditions treatable by these therapeutically active agents.

The present invention, in some embodiments thereof, relates to a methodology for encapsulating active agents in an amphiphilic polymer comprising various hydrophobic and hydrophilic domains within the same particle. The polymer may have nano-sized or submicron-sized structure. Using this methodology, polymeric structures encapsulating drugs have been prepared and characterized, and are further shown to associate to the drug with high affinity thereto.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Compositions-of-Matter

According to an aspect of some embodiments of the present invention, there is provided a composition-of-matter comprising one or more amphiphilic co-polymers, the amphiphilic co-polymers comprising one or more hydrophobic domains and one or more hydrophilic domains, wherein each of the hydrophobic domains and the hydrophilic domains comprise a polymeric backbone; and wherein the polymeric backbone of the hydrophobic domain is attached to the polymeric backbone of the hydrophilic domain.

In some embodiments, there is provided a composition-of-matter comprising one or more amphiphilic block or graft polymers, one or more amphiphilic block or graft polymers comprising hydrophobic domain and a hydrophilic domain, wherein:
each of the hydrophobic domain and the hydrophilic domain comprises a polymeric backbone having at least two monomeric units;
the polymeric backbone of the hydrophobic domain is attached to the polymeric backbone of the hydrophilic domain; and
the one or more amphiphilic block polymers are configured to form a multi-micellar structure, at a critical micellar concentration (CMC) of below than 4% w/v.

In some embodiments, the amphiphilic co-polymer is a block co-polymer. In some embodiments, the amphiphilic co-polymer is a graft co-polymer. In some embodiments, the amphiphilic block or graft copolymers (referred to as “block”) may form a structure having a hydrophobic core and a hydrophilic corona. In some embodiments, the amphiphilic block or graft copolymers may form various hydrophobic domains and various hydrophilic domains, wherein at least portion of the hydrophilic domain is capable of binding carbohydrate cell receptors and/or transporters expressed at the outer surface of the cellular membrane.

In some embodiments, the hydrophobic blocks are configured to form a hydrophobic core and the hydrophilic blocks are configured to form a hydrophilic corona.

In some embodiments, the hydrophobic blocks are configured to form a plurality of hydrophobic domains and the hydrophilic blocks are configured to form a plurality of hydrophilic domains.

In some embodiments, each of the hydrophobic domains and hydrophilic domains comprise at least two monomeric units, and the hydrophobic domain is attached to a backbone of the hydrophilic domain.

In some embodiments, the hydrophobic domain is present at a concentration of 2%, 5%, 10%, 15%, 20%, 25%, 30%, or 35%, by weight, including any value and range therebetween.

By “attached to”, also referred to herein as “grafted to”, it is meant to refer to covalently bound, conjugated, hybridized, or immobilized.

As used herein throughout, the term “polymer” describes an organic substance composed of a plurality of repeating structural units (backbone units) covalently connected to one another.

Herein throughout, the term “monomer” refers to a molecule that may bind chemically to other molecules to form an oligomer or a polymer.

The term “monomeric unit” refers to the repeating units, derived from the corresponding monomer. The terms “repeating unit” and “monomeric unit” are used herein throughout interchangeably. The polymer comprises the monomeric units. By “derived from” it is meant to refer to the polymeric compound following the polymerization process.

In some embodiments, the polymeric backbone of each of the hydrophobic domains comprises two or more monomeric units.

In some embodiments, the polymeric backbone of each of the hydrophilic domains comprises two or more monomeric units.

The term “amphiphilic polymer” is understood to mean a polymer which comprises at least a hydrophilic part (the term “part” is also referred to herein throughout as “block”, “domain” or “component”, interchangeably) and at least a hydrophobic part. This polymer is water-soluble or water-dispersible, directly or e.g., by means of pre-dissolution in an organic solvent miscible with water or a solvent that may be eliminated before redispersion of the amphiphilic polymer in water.

The polymers disclosed herein can be block or graft copolymers which comprise, on the one hand, at least one water-soluble or water-dispersible polymer block and, on the other hand, at least one hydrophobic block.

The term “block copolymer” refers to copolymers wherein monomeric units of a given type are organized in blocks, i.e. monomeric units of the same type are adjacent to each other. To explain further, the term “block copolymer” includes molecules of the type AᵢBⱼAₖ, wherein A and B designate distinct types of monomers and the indices i, j, k and l are integer numbers having a value of at least 1.

The term “amphiphilic block copolymer” according to some embodiments of the present invention designates block copolymers, comprising or consisting of a hydrophobic part and a hydrophilic part, wherein either or both parts may be made of one or more types of monomeric units, the monomeric units being organized in blocks. For example, the term “amphiphilic block copolymer” may relate to di-block copolymers of the general formula AᵢBⱼ, wherein one of Aᵢ or Bⱼ is a hydrophobic polymer and the respective other moiety is a hydrophilic polymer.

The term “amphiphilic block copolymer” according to some embodiments of the present invention designates block copolymers, comprising or consisting of a hydrophobic part (domain) and a hydrophilic part (domain), wherein either or both parts may be made of one or more types of monomeric units, the monomeric units being organized in blocks.

The term “graft copolymer” refers to a copolymer having a backbone or main chain, side chains of different chemical groups at different positions connected along the backbone to the backbone or main chain. The side chains can be incorporated at different positions along the backbone by covalent attachment, to form the graft copolymer.

The terms “hydrophilic”, “water-soluble”, and “water-dispersible” are used herein throughout interchangeably.

The polymers employed in the context of the present invention can thus be, in some embodiments, block (or multiblock) or graft copolymers comprising, for example, hydrophilic domains alternating with hydrophobic blocks. These polymers can also be provided in the form of grafted polymers, the backbone of which is composed of water-soluble or water-dispersible blocks and carries hydrophobic grafts of variable chemical composition and number of repeating units. In some embodiments, the backbone of the grafted polymers (e.g., water-soluble or water-dispersible block) is characterized by high affinity to cell via a carbohydrate determinant, carbohydrate-specific receptors, or transporter (e.g., glucose transporter) exposed at the outer surface of the cell membrane with different selectivity and efficacy.

In some embodiments by “affinity” it is meant to refer to facilitating the interaction of the resulting conjugate of the disclosed composition with carbohydrate-specific receptor biomolecule, or in some embodiments, adheres or binds to a specific target bearing a receptor protein which may bind a saccharide moiety and thus, in some embodiments, may enable the recognition and further trafficking of the composition to target tissues, cells or organelles and/or its uptake across cellular membranes via interaction with the specific membranal receptor.

Typically, but not exclusively, the term “hydrophilic domain” may be understood to mean a polymer which, when introduced into water at a concentration equal to 1%, by weight, results in a macroscopically homogeneous solution.

In some embodiments, the transmission of light at a wavelength equal to 500 nm through a sample of the disclosed composition having a thickness of 1 cm, is at least at least 5%, 10%, or at least 15%, which corresponds to an absorbance [abs=−log(transmission)] of e.g., less than 1.5.

Typically, but not exclusively, the term “amphiphilic polymer” may be understood to mean a polymer which, upon introducing into aqueous solution at 0.05% (by weight), makes it possible to reduce the surface tension of the water at 25° C. to a value of less than 50 mN/m, e.g., less than 40 mN/m.

The amount (as active material) of the amphiphilic polymer in the composition of the invention may range from e.g., 0.0001% to 30%, by weight, with respect to the total weight of the composition.

In some embodiments, the weight ratio of the hydrophobic domain to the total weight of the amphiphilic polymer ranges from 1 to 70% or, in some embodiments, from 10 to 50%.

### Hydrophilic Domains

Mention may be made, by way of example and without being limited thereto, of the following water-soluble monomers and their salts which are capable of being employed to form the water-soluble or water-dispersible domains, blocks or units, alone or in the form of a mixture thereof: alginate, galactomannan, hydrolyzed galactomannan, glucomannan, hydrolyzed glucomannan, guar gum, xanthan gum, pectin, cellulose, nanocrystalline cellulose, dermatan sulfate, cyclodextrins, poly(cyclodextrins), dextran, dextrin, starch, hyaluronic acid, chondroitin 4-sulfate, chondroitin 6-sulfate, heparin, heparan sulfate, keratin sulfate, beta-glucan, fucoidan, mannan, fucomannan, galactofucan, glucofucan and levan.

In some embodiments, the hydrophilic component of the disclosed amphiphilic block polymer comprises a multifunctional polymer.

In some embodiments, the hydrophilic component of the disclosed amphiphilic block polymer is selected from natural, modified natural, synthetic or semisynthetic polymers.

Non-limiting examples of hydrophilic polymers may be e.g., natural, synthetic or semisynthetic polyols, polycarboxylic acids, polysulfates, polyamines, polysaccharides, poly(cyclodextrins).

As used herein, functional groups, include, but are not limited to, a hydroxyl group, an amine group, a thiol group, a carboxyl group, a keto group, a sulfate group, a double or triple bond group, or any other reactive functional group, and combinations thereof.

In exemplary embodiments, the hydrophilic domain comprises monomeric units (or the corresponding polymeric domain derived therefrom) selected from, without limitation, glucose (forming cellulose, nanocrystalline cellulose, poly(cyclodextrins), dextran, dextrin, starch, guluronic and mannuronic acid (forming alginate), galactose and mannose (forming galactomannan or hydrolyzed galatomannan); galactose and mannose (forming guar gum); glucose and mannose (forming glucomannan or hydrolyzed glucomannan); glucose, mannose, and glucuronic (forming xanthan gum), galacturonic acid (forming pectin); glucuronic acid (forming hyaluronic acid), N-acetylgalactosamine and glucuronic acid (forming chondroitin 4-sulfate and chondroitin 6-sulfate); N-acetylgalactosamine and glucuronic acid (forming dermatan sulfate); beta-glucose (forming beta-glucan), fructose (forming fucoidan), mannose (forming mannan), fructose and mannose (forming fucomannan), galactose and fucose (forming galactofucan) or glucose and fucose (forming glucofucan), or any combination thereof.

In some embodiments, the hydrophilic domain comprises galactomannan, wherein the ratio of galactose units to mannose units ranges from 1:1 to 1:20, from 1:1 to 1:10, from 1:1 to 1:5.

In some embodiments, the hydrophilic domains may have a molar mass of e.g., between 1000 g/mol and 10,000,000 g/mol when they constitute the water-soluble backbone of the block polymer. In some embodiments, these water-soluble blocks have a molar mass of between 1000 g/mol and 10,000 g/mol upon constituting a block or multiblock polymer.

### Hydrophobic Domains

Typically, but not exclusively, the term “hydrophobic domain” is understood to mean blocks which are soluble or dispersible in fatty substances which are liquid at ambient temperature (e.g., 25° C.) or oils, such as alkanes, esters, ethers, triglycerides, silicones or fluorinated or other halogenated compounds or a mixture of hydrophobic materials (oils).

The terms “hydrophobic” and “water-insoluble” are used herein throughout interchangeably. The term, “water-insoluble” is defined to mean that less than e.g., 5 g, 4 g, 3 g, 2 g, 1 g, 0.5 g, 0.4, g, 0.3 g, 0.2 g, or less than 0.1 g of the domain is soluble in 100 g of water.

In some embodiments, the hydrophobicity characteristic of the domain is unchanged regardless of temperature (e.g., 20° C. to 40° C.). In some embodiments, the hydrophobicity characteristic of the domain is maintained at a defined range of temperature (e.g., 20° C. to 40° C.).

In some embodiments, the hydrophobicity characteristic of the domain is unchanged regardless of the pH (e.g., 5 to 9). In some embodiments, the hydrophobicity characteristic of the domain is maintained at a defined range of pH (e.g., 5 to 9).

In some embodiments, the hydrophobic domain may comprise one or more blocks of a hydrophobic molecule or polymer such as, without limitation, lipidic molecules, lipids, phospholipids, or any other linear or branched polymer or oligomer.

In exemplary embodiments, the hydrophobic domain comprises or is derived from, without being limited thereto, Poly(aspartic acid), Poly(β-benzyl-L-aspartate), Poly(epsilon-caprolactone) (PCL), Poly(D,L-lactide), Poly(propylene oxide) (PPO), Poly(methyl acrylate), and Poly(methyl methacrylate), or any derivative or block polymer thereof (e.g., Poly(butylene oxide) (PBO) and poly(propylene oxide) (PPO) block co-polymers).

In exemplary embodiments, the hydrophobic domain is derived from methyl methacrylate (MMA) or a polymer thereof.

In exemplary embodiments, the composition comprises poly(methyl methacrylate) (PMMA) grafted with polysaccharide such as galactomannan. In exemplary embodiments, the total PMMA (w/w) content is 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%, including any value and range therebetween. In exemplary embodiments, the total weight ratio of polysaccharide such as galactomannan to PMMA is 2:1 to 50:1, or 3:1 to 40:1, respectively, including any value and range therebetween.

In some embodiments, the polymer is thermo-responsive polymer characterized by a defined lower critical solution temperature (LCST). For example, copolymers of N-isopropylacrylamide (NIPAAm) and acrylamide (AAm) exhibit a LCST that is slightly below body temperature (30-35° C.). Below the LCST the polymer undergoes hydration and thus it is water-soluble, whereas above the LCST hydrophobic interactions begin to appear, followed by de-hydration and shrinkage.

In some embodiments, the hydrophobic domain is a block with at least two repeating units of any polyester, polyether, polycarbonate, polyanhydride, polyamide, polyacrylate, polymethacrylate, or any other hydrophobic homopolymer or heteropolymer, or a mixture thereof or any hydrophobic molecule of a group of fatty acids, fatty alcohols, or any other lipid molecule with at least two carbons in the backbone that may be grafted to the hydrophilic multifunctional polymer through the reactive functional groups.

In some embodiments, the molar mass of these hydrophobic blocks is between 100 g/mol and 10,000 g/mol. In some embodiments, the molar mass is between 200 g/mol and 5,000 g/mol.

Amphiphilic Polymers

In some embodiments, the hydrophobic component of the materials of this invention is directly copolymerized to the hydrophilic polymer component by means of any copolymerization (also referred to as “condensation”) reaction known in the art (e.g. graft-polymerization).

Non-limiting examples of such condensation reaction are selected from coordination polymerization, ring-opening polymerization, free radical polymerization, living polymerization and any other methodology of graft polymerization.

By “living polymerization” it is meant to refer to a form of chain growth polymerization where the ability of a growing polymer chain to terminate has been removed. Living radical polymerization is a type of living polymerization where the active polymer chain end is a free radical.

Living polymerization may be selected from living cationic polymerization, living anionic polymerization, and atom-transfer radical (ATR) polymerization.

Several methodologies of living radical polymerization are known in the art and are conceivable to be applied in the context of the present invention, including, without limitation, reversible-deactivation polymerization, catalytic chain transfer, cobalt mediated radical polymerization, iniferter polymerization, stable free radical mediated polymerization, ATR, reversible addition fragmentation chain transfer (RAFT) polymerization, iodine-transfer polymerization (ITP), selenium-centered radical-mediated polymerization, telluride-mediated polymerization (TERP), and stibine-mediated polymerization.

The term “condensation reaction”, also referred to in the art as “step-growth process”, and the like, means reaction to form a covalent bond between organic functional groups possessing a complementary reactivity relationship, e.g., electrophile-nucleophile. Typically, the process may occur by the elimination of a small molecule such as water or an alcohol. Additional information can be found in G. Odian, Principles of Polymerization, 3rd edition, 1991, John Wiley & Sons: New York, p. 108.

In some embodiments, the hydrophobic component is copolymerized directly to the hydrophilic polymer component without a coupling agent.

In some embodiments, the hydrophobic component is copolymerized to the hydrophilic polymer component by a coupling agent. In some embodiments, the hydrophobic component is not copolymerized to the hydrophilic polymer component via a coupling agent.

In some embodiments, the polymeric backbone of the hydrophobic domain is attached to a side-chain group of the polymeric backbone of the hydrophilic domain via a bifunctional coupling agent selected from the group consisting of: diisocyanates, disilanes, dialkoxysilanes, diglycidyl ethers, or any other bifunctional coupling agent or any other coupling agent with a higher functionality than two or any condensation agent such as dicyclohexylcarbodiimide, O-(benzotriazol-1-yl)-N,N,N′,N′-tetramethyluronium tetrafluoroborate, and combinations thereof.

In some embodiments, the coupling agent is a bifunctional coupling agent.

In some embodiments, the coupling agent is a multifunctional coupling agent.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains or between domains within a polymeric structure, or between other chemically compatible functional groups of polymeric chains.

In some embodiments of the present invention, the term “coupling agent” is referred to as “crosslinking agent”. In some embodiments, one of the domains serves as the coupling agent or as a crosslinking polymer.

Non-limiting examples of coupling agents include diisocyanates, disilanes, dialkoxysilanes, diglycidyl ethers, or any other bifunctional coupling agent or any other coupling agent with a higher functionality than two or any condensation agent such as dicyclohexylcarbodiimide, O-(benzotriazol-1-yl)-N,N,N′,N′-tetramethyluronium tetrafluoroborate, and combinations thereof.

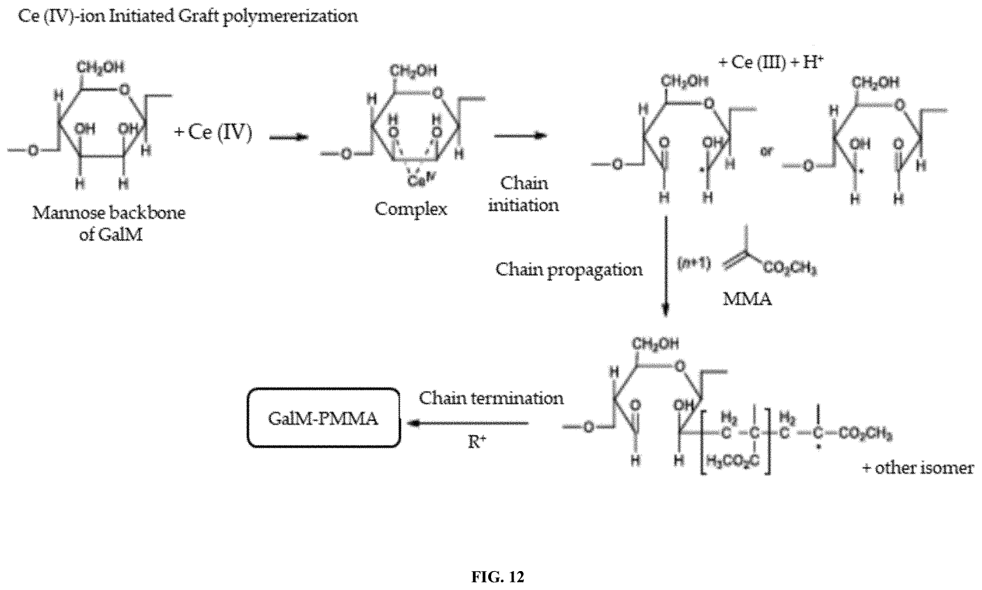

In some embodiments, the hydrophobic domain is grafted to a backbone of the hydrophilic domain. In some embodiments, the hydrophobic domain is grafted to a side-chain of the hydrophilic domain. In some embodiments, the hydrophobic domain is grafted via a C—C bond, thereby forming a side chain attached to the backbone of the hydrophilic domain. In some embodiments, the hydrophobic domain is grafted to a mannose unit. Such grafted polymeric structure is exemplified by FIG. 12.

As noted hereinabove, the hydrophilic blocks may form a corona structure, wherein in some embodiments, the hydrophilic corona binds carbohydrate cell receptor and/or transporter expressed at the outer surface of the cellular membrane. As further noted hereinabove, the hydrophobic blocks may form a core structure or various forms of hydrophobic domains.

As noted hereinabove, the hydrophilic blocks may form a plurality of hydrophilic domains, wherein in some embodiments, the hydrophilic corona binds carbohydrate cell receptor and/or transporter expressed at the outer surface of the cellular membrane. As further noted hereinabove, the hydrophobic blocks may form a core structure or form various hydrophobic domains.

In some embodiments, the hydrophobic domain is in the range of 10% to 90%, by weight, of the amphiphilic block polymer.

In some embodiments, the amphiphilic block polymer(s) is characterized by a hydrophilic-lipophilic balance (HLB) value that ranges from 1 to 24.

In some embodiments, the amphiphilic block polymer(s) is characterized by a HLB value that ranges from 4 to 15.

In some embodiments, the amphiphilic block polymer(s) is characterized by an average molecular weight ranging from 5000 to 2,000,000 g/mol.

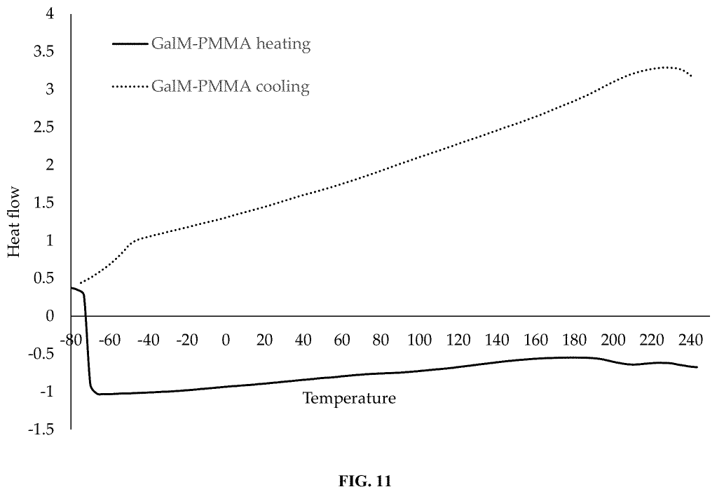

In some embodiments, the amphiphilic block polymer(s) is characterized by a glass transition temperature (T_{g}) ranging from −60 to 200° C. An exemplary differential scanning calorimetry (DSC) curve, showing a Tg of an amphiphilic block polymer is presented in FIG. 11.

As in all associative chemical reactions, the formation of a bond occurs between two groups within compounds or compositions depending on sufficient proximity there between. In the context of some embodiments of the present embodiments, the degree of sufficient proximity depends on the attractive forces that can be exerted by the associating groups and the relative reactivity thereof.

The phrase “attractive force”, as used herein, refers to physical forces that span and have an effect over a distance, or field, such as electric and magnetic fields. Associating groups which can exert an attractive force field may attract each other over a definable distance, such as in the case of atoms having electrostatic charges.

The term “proximity” as used herein therefore describes any distance that allows interaction between such associating groups, whereby this distance can be practically null and depends on the presence, type and extent of the attractive forces which can be exerted by and affect the associating groups.

A pair of associating groups on two monomeric units or domains should also be oriented appropriately so as to allow a constructive encounter there between which results in the formation of a chemical bond. This is particularly important in cases where the associating groups are characterized by radial asymmetry, directivity, polarity, dipole, vectorial force, effective angle and/or other directional and spatial characteristics. An appropriate orientation is determined by steric constrains, surface accessibility and other structural complementarity considerations as described hereinabove. The term “orientation” therefore refers to a steric location and directionality of an object with reference to another object (herein the associating groups).

Regardless if the associating groups exert an attractive force field which extends beyond the physical boundary of the monomeric units or domains, or the degree of mutual reactivity of the associating groups, the monomeric units or domains must be subjected to suitable conditions which will allow them to associate there between. By suitable conditions it is meant that the monomeric units or domains need to be present at an adequate density (concentration) and possess suitable kinetic energy (temperature) so as to produce a sufficient number of events in which the monomeric units or domains come in contact in the chemical sense, interact and associate (joined together). By “interact” it is meant that one or more monomeric units or domains, each having associating groups thereon, while being subjected to suitable conditions as discussed herein below, can come close enough to one another, and at a certain angle range, so as to allow the associating groups to be attached to one another and/or to self-assemble.

In addition to an adequate concentration and suitable temperature, the condition which allows the self-assembly of a chemical structure includes other factors which affect the chemical environment in which the monomeric units or domains are placed. These factors include the type of medium (solvent), the ionic strength and pH of the medium (solutes and buffers) and the presence of other chemical agents such as catalysts, oxidation and reduction agents, and other factors which may affect the reactivity of the associating groups.

The terms “self-assembled”, or “self-aggregated”, refer to a resulted structure of a self-assembly process based on a series of associative chemical reactions between at least two chemical domains or polymers, which occurs when the associating groups on one chemical domains or polymers are in sufficient proximity and are oriented so as to allow constructive association with another domain or polymer. In other words, an associative interaction means an encounter that results in the attachment of the domains or the polymers to one another.

Closed, hollow and self-assembled chemical structures as described herein below (“self-assembled core-corona structures” or “self-assembled multi-micellar structures”) may be used in a myriad of applications, owing to several of the following most consistent and unique characteristics, such as:
capacity to assemble and optionally disassemble under particular chemical and physical conditions;
hollow and closed interior;
uniform and reproducible distribution of shape, size and composition;
defined (e.g., spherical, disc, cylindrical) overall shape; and
wide range of controllable sizes.

One of the most intuitive uses of a closed and hollow molecular core that can reversibly self-assemble is a vehicle for substance retention, and subsequent release thereof in or to a chemical, biological or physiological system. Other uses of the closed and hollow structures described herein may utilize the unique structural features of the disclosed amphiphilic block polymer delineated herein throughout. Further embodiments of this aspect are detailed herein below under “Pharmaceutical Composition”.

As mentioned hereinabove, in some embodiments, the composition-of-matter is in form of a closed, e.g., hollow, and self-assembled structure, the structure having a hydrophobic core and a hydrophilic corona, wherein the hydrophilic corona is layered in a non-covalent manner. As abovementioned, in some embodiments, the term “layer”, or any grammatical derivative thereof, refers to a non-covalent crosslinking structure. In some embodiments, the term “layer”, or any grammatical derivative thereof, refers to covalent crosslinking structure. In some embodiments the hydrophilic corona interacts with an aqueous solution, thus stabilizing the self-assembled structure in the solution. In some embodiments water molecules form non-covalent interactions with hydrophilic corona, resulting in a non-covalently crosslinked polymeric matrix.

In some embodiments, the composition-of-matter comprises a plurality of self-assembled amphiphilic polymers. In some embodiments, the plurality of the amphiphilic polymers is self-assembled.

In some embodiments, the amphiphilic block polymer described herein or the composition-of-matter comprising the same are in form of solid particles.

In some embodiments, a plurality of amphiphilic block or graft polymers described herein forms a micelle, a micelle-like, a core-corona structure, or a multi-micellar structure.

As used herein, the term “micelle” describes a colloidal particle, in a simple arrangement or geometric form, typically spherical, of a specific number of amphiphilic molecules, which forms at a well-defined concentration, called the critical micellar concentration (CMC). The micelle can be a single particle or can be formed by a cluster of several micelles, which interact with one another so as to form a particle having a larger dimension (referred to as “multi-micellar particle”).

The phrase “critical micellar concentration” (CMC) describes the concentration of the disclosed amphiphilic block copolymers above which the disclosed amphiphilic block copolymers are present substantially in a micellar form under a given set of conditions. At the vicinity of CMC, sharp change in many experimental parameters may be observed, and this may be measured by a number of techniques that include, but not limited to, surface tension measurements, fluorescence, light scattering, conductivity, osmotic pressure, and the like CMC varies as a function of a number of physical factors such as pH, temperature, ionic strength and pressure.

In some embodiments, the disclosed amphiphilic block copolymer or a plurality thereof form a closed, e.g., hollow, and self-assembled structure e.g., micelle or a micelle-like structure. That is, each self-assembled structure comprises at least e.g., 1, 2, 3, 4, 5, 6, 7, 8, 9, 10, 11, 12, 13, 15, 16, 17, 18, 19, 20, 30, 40, 50, 60, 70, 80, 90, 100, 1000, 10,000, or at least 100,000 amphiphilic block copolymers.

As used herein, the terms “corona”, or “shell”, which are used herein throughout interchangeably, refer to the sphere (typically the hydrophilic domain(s)) surrounding the core. The term “sphere” is used only for the purpose of illustration and it is to be construed that is not only limited to spherical shape but also includes any shape which may find suitability to at least some embodiments of the present invention.

The term “core” refers to the central region (typically the hydrophobic domain(s)) of the structure, which typically contains the hollow.

The term “hydrophilic domain” may refer to zones in the particle that contain mainly (e.g., at least 60% or at least 70%) the hydrophilic block of the polymer.

The term “hydrophobic domain” may refer to zones in the particle that contain mainly (e.g., at least 60% or at least 70%) the hydrophobic blocks of the polymer.

The term “closed” as used herein, is a relative term with respect to the size, the shape and the composition of two entities, namely an entity that defines an enclosure (the enclosing entity) and the entity that is being at least partially enclosed therein. In general, the term “closed” refers to a morphological state of an object which has discrete inner and outer surfaces which are substantially disconnected, wherein the inner surface constitutes the boundary of the enclosed area or space. The enclosed area or space may be secluded from the exterior area of space which is bounded only by the outer surface.

In the context of the present invention, the closure of the enclosing entity depends of the size, shape and chemical composition of the entity that is being enclosed therein, such that the enclosing entity may be “closed” for one entity and at the same time be “open” for another entity. For example, structures presented herein are closed with respect to certain chemical entities which cannot pass through their enclosing shell or corona, while the same “closed” structures are not closed with respect to other entities.

For example, the structures of the present embodiments may be closed with respect to, for example, a drug molecule, but non-closed with respect to, for example, a single atom ion or an atom of a noble gas. In the context of the present invention, the same “closed” structures are affected by certain conditions e.g., pH, temperature, concentration, etc.

The terms “hollow” or “hollow sphere” is used only for the purpose of illustration and it is to be construed that is not only limited to spherical shape but also includes any shape which may find suitability to at least some embodiments of the present invention, the same “closed” structures are affected by certain conditions e.g., pH, temperature, concentration, etc.

The term “hollow”, as used herein, refers to an object having a vacuous cavity, a gap, a void space or an empty space enclosed within. The term “hollow” is not only limited to spherical shape but also includes any shape which may find suitability to at least some embodiments of the present invention. By “void space” herein it is meant to refer to a polymer-free space or a central cavity. The term hollow is used herein as an illustration and it is to be construed that the core-corona structure are not fully hollow in the core.

In some embodiments, the core or the various hydrophobic domains are of e.g., spherical, cylindrical, rod, lamellae, irregular or any other morphology.

In some embodiments, the core-corona structure (e.g., micelle) of the disclosed composition remains stable above specific pH condition and/or specific concentration (e.g., CMC).

In some embodiments, the multi-micellar structure of the disclosed composition remains stable above specific pH condition and/or specific concentration (e.g., CMC).

Typically, but not exclusively, the CMC has a value of e.g., 1×10⁻⁵% w/v to 1×10⁻²% w/v.

In some embodiments, the multi-micellar structure of the one or more amphiphilic block copolymers remains stable within a specific range of temperature.

In some embodiments, the hydrophobicity characteristic of the domain (e.g., a hydrophobic domain comprising methyl methacrylate monomeric units) is substantially not temperature- and/or pH-dependent.

In some embodiments, once solubilized in water or in any other aqueous medium, and at a final concentration above a certain concentration and/or at certain range of temperature, the amphiphilic block polymer(s) of the disclosed composition undergo self-aggregation to form nanoscopic, submicroscopic or microscopic structures.

As used herein throughout, the term “stable”, or any grammatical derivative thereof, may refer to chemical stability. “Chemical stability” means that an acceptable percentage of degradation of the self-assembled structure disclosed herein throughout produced by chemical pathways such as oxidation or hydrolysis is formed. In particular, the self-assembled structure is considered chemically stable if no more than about 10% breakdown products are formed after e.g., two weeks of storage at the intended storage temperature of the product (e.g., room temperature, i.e. 15° C. to 30° C.).

The term “stable”, or any grammatical derivative thereof, may also refer to physical stability. The term “physical stability” means that with respect to the self-assembled structure disclosed herein throughout, an acceptable percentage of aggregates (e.g., dimers, trimers and larger forms) remains formed. In particular, a formulation is considered physically stable if at least about 15% of the aggregates remain formed after e.g., two weeks of storage at the intended storage temperature of the product (e.g., room temperature).

The term “stable” may also refer to the active or therapeutic agent (as described herein below) encapsulated within the self-assembled structure, meaning that at least about 65% of therapeutic agent remains chemically and physically stable after e.g., one month of storage at room temperature.

In some embodiments, at least 70% of the self-assembled structures are characterized by a hydrodynamic size (also referred to herein throughout as “hydrodynamic diameter” or, for simplicity, “size” or “diameter”) that ranges from 10 nm to 10 μm. In some embodiments, at least 80% of the particles are characterized by a hydrodynamic size that ranges from 10 nm to 10 μm. In some embodiments, at least 90% of the self-assembled core-corona structures are characterized by a hydrodynamic size that ranges from 10 nm to 10 μm.

In some embodiments, at least 80% of the self-assembled core-corona structures are characterized by a hydrodynamic size that ranges e.g., from 10 nm to 10 μm, or from 10 nm to 1 μm, or from 20 nm to 500 nm, or from 50 nm to 100 nm, or from 10 nm to 50 nm, or from 100 nm to 1 μm. In exemplary embodiments, the hydrodynamic size ranges from about 300 nm to about 500 nm.

In some embodiments, a plurality of self-assembled core-corona structures as disclosed herein is characterized by a narrow hydrodynamic size distribution.

As used herein “narrow hydrodynamic size distribution” is characterized by e.g., at least 60%, at least 70%, at least 80%, at least 90%, of the particles having a hydrodynamic size that varies within a range of less than 25%.

In some embodiments, the “narrow hydrodynamic size distribution” is characterized by size distribution of at least 80% of the particles varying within a range of less than e.g., 60%, 50%, 40%, 30%, 20%, 10%, including any value there between.

In some embodiments, the “narrow hydrodynamic size distribution” is characterized by size distribution of at least 80% of the self-assembled core-corona structures varying within a range of less than e.g., 60%, 50%, 40%, 30%, 20% or 10%.

As described hereinabove, in some embodiments of the present invention, the hydrophilic corona has a high affinity to carbohydrate cell receptor and/or transporter (e.g., glucose transporter) expressed at the outer surface of a cell membrane with different selectivity and efficacy.

By “cell receptor” or “transporter” it is meant to refer to any membrane or transmembrane protein expressed at any cell surface (e.g., outer cell surface, nucleus membrane, mitochondrial membrane) that selectively recognizes any of the repeating units of the hydrophilic blocks of the polymer or clusters of repeating units as a substrate and may bind it with variable selectivity and affinity.

In some embodiments of the present invention, the cell receptors are selected from a group of lectin-like receptors, mannose receptor, fructose receptor, galactose receptor, dermatan sulfate receptor, cluster of differentiation 44 (CD44), exposed at the outer surface of the cell membrane or any other cell membrane (e.g., nucleus membrane).

In some embodiments of the present invention, the cell transporters are selected from glucose transporters, exposed at the outer surface of the cell membrane or any other cell membrane (e.g., nucleus membrane).

In some embodiments, the amphiphilic block or graft polymer or the composition-of-matter comprising thereof is biodegradable. In some embodiments, the amphiphilic block polymer or the composition-of-matter comprising thereof is not biodegradable. In this context, the “amphiphilic block or graft polymer or the composition-of-matter comprising thereof” may refer to hydrophilic component of the composition-of-matter and/or the hydrophobic component.

As used herein, the term “biodegradable” describes a substance which can decompose under physiological and/or environmental conditions into breakdown products. Such physiological and/or environmental conditions include, for example, hydrolysis (decomposition via hydrolytic cleavage), enzymatic catalysis (enzymatic degradation), and mechanical interactions. Typically, but not exclusively, this term refers to substances that decompose under these conditions such that e.g., 50 weight percent of the substance decompose within a time period shorter than one year.

The term “biodegradable” as used in the context of embodiments of the invention, also encompasses the term “bioresorbable”, which describes a substance that decomposes under physiological conditions to break down products that undergo bioresorption into the host-organism, namely, become metabolites of the biochemical systems of the host-organism.

In some embodiments, at least e.g., 50%, 60%, 70%, 80%, 90% or 99% of plurality of the disclosed amphiphilic block polymers is characterized by a low dispersity index (D). Herein, the “disclosed amphiphilic block or graft polymers” may refer to the amphiphilic block polymers prior to their non-covalently bonding, or, in some embodiments, following the non-covalently bonding as described herein throughout.

As used herein, “dispersity index”, also termed in the art: “polydispersity index” (denoted herein throughout as: “D” or “PDI”) refers to a measure of the distribution of molecular mass in a given polymer sample. The dispersity index is calculated by dividing the weight average molecular weight (Mw) by the number average molecular weight (Mn). As used herein, the term “weight average molecular weight” generally refers to a molecular weight measurement that depends on the contributions of polymer molecules according to their sizes. As used herein, the term “number average molecular weight” generally refers to a molecular weight measurement that is calculated by dividing the total weight of all the polymer molecules in a sample with the total number of polymer molecules in the sample. These terms are known by those of ordinary skill in the art.

D has a value always greater than 1, but as the polymer chains approach uniform chain length, the value of D approaches unity (1). Homogenous size distribution of the amphiphilic block polymers may contribute, inter alia, to a more defined biodistribution.

As used herein “low D value” refers to a value below 3. For example a “low D value” may be 2.99, 2.98, 2.97, 2.96, 2.95, 2.94, 2.93, 2.92, 2.91, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.19, 1.18, 1.17, 1.16, 1.15, 1.14, 1.13, 1.12, 1.11, 1.1, 1.09, 1.08, 1.07, 1.06, 1.05, 1.04, 1.03, 1.02, or 1.01, including any value therebetween.

In dynamic light scattering (DLS), the (absolute) width of the distribution can be compared to the mean, and a relative DLS-PDI=width/mean is obtained. For a theoretical Gaussian distribution the overall polydispersity is the relative polydispersity of the distribution. Traditionally, this overall polydispersity has also been converted into an overall DLS-PDI which is the square of the light scattering polydispersity. For a perfectly uniform sample, the PDI value in the context of DLS would be 0. As demonstrated in the Examples section that follows, in exemplary embodiments, the DLS-PDI has values in the range of e.g., about 0.01 to about 0.5, about 0.02 to about 0.2, or from about 0.05 to about 0.1

In some embodiments, a plurality of self-assembled core-corona structures as disclosed herein is characterized by a negative zeta-potential. In some embodiments, zeta-potential has a value ranging from −1 to −10 mV, from −1 to −2 mV, from −2 to −4 mV, from −4 to −7 mV, from −7 to −10 mV, from −6 to −7 mV, or any range there between.

In some embodiments, a plurality of self-assembled core-corona structures as disclosed herein is characterized by a positive zeta-potential. In some embodiments, zeta-potential has a value ranging from 1 to 10 mV, from 1 to 2 mV, from 2 to 4 mV, from 4 to 7 mV, from 7 to 10 mV, from 6 to 7 mV, or any range there between.

### Pharmaceutical Compositions

According to another aspect of the present invention, there is provided a composition and method of preparing a composition which comprises a core-corona e.g., self-assembled, chemical structure of the amphiphilic block copolymer(s) described hereinabove, and an active agent or otherwise a substance being encapsulated in the chemical structure e.g., within the core.

In some embodiments, the hydrophobic block of the amphiphilic block copolymer stabilizes the core. In some embodiments, the hydrophobic block encapsulates the core. In some embodiments, the core comprises an active agent. In some embodiments, the core comprises a solution of the active agent. In some embodiments, the core comprises a dispersion of the active agent. In some embodiments, the hydrophobic block bounds the active agent. In some embodiments, the active agent interacts non-covalently with the hydrophobic block. In some embodiments, the hydrophobic block encapsulates the active agent.

In some embodiments, the active agent is hydrophobic. In some embodiments, the active agent is oil soluble. In some embodiments, the active agent is oil insoluble.

In some embodiments, an active agent is bound to the hydrophilic corona. In some embodiments, an active agent is non-covalently bound to the corona. In some embodiments, an active agent is bound to the corona via hydrogen bonding. In some embodiments, an active agent is bound to the corona via electrostatic interactions. In some embodiments, an active agent is bound to the corona via a covalent bond.

In some embodiments, an active agent is bound to the hydrophobic core. In some embodiments, an active agent is non-covalently bound to the core. In some embodiments, an active agent is bound to the core via hydrophobic interactions. In some embodiments, an active agent is bound to the corona via van der Waals. In some embodiments, an active agent is bound to the core via covalent bond.

In some embodiments, an active agent bound to the corona is hydrophilic (e.g. DNA, RNA or a peptide). In some embodiments, the active agent is water-soluble (e.g. a water-soluble drug). In some embodiments, the active agent bound to the corona is protected from degradation (e.g. intracellular or extracellular). In some embodiments, the hydrophilic active agent bound degradation to the corona of the self-assembled or micellar like structure has an enhanced stability in-vivo. In some embodiments, the self-assembled or micellar like structure is used for targeted delivery of the hydrophilic active agent. In some embodiments, the self-assembled or micellar like structure is used for controlled release of the hydrophilic active agent.

In some embodiments, an active agent bound to the core is hydrophobic (e.g. dasatinib, imatinib). In some embodiments, the active agent bound to the core is protected from degradation (e.g. intracellular or extracellular). In some embodiments, the hydrophobic active agent bound to the corona of the self-assembled or micellar like structure has an enhanced stability in-vivo. In some embodiments, the self-assembled or micellar like structure is used for targeted delivery of the hydrophobic active agent. In some embodiments, the self-assembled or micellar like structure is used for controlled release of the hydrophobic active agent.

In some embodiments, the composition comprises about e.g., 0.5%, 1%, 1.5%, 2%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 20%, 30%, or about 40% of active agent, including any value and range there between, by total dry weight of the composition.

As used herein the terms “pharmaceutical composition” or “pharmaceutical product”, which are used herein throughout interchangeably, refers to a preparation of one or more of the compositions described herein, or physiologically acceptable salts or prodrugs thereof, with other chemical components including but not limited to physiologically suitable carriers, excipients, lubricants, buffering agents, antibacterial agents, bulking agents (e.g. mannitol), antioxidants (e.g., ascorbic acid or sodium bisulfite), anti-inflammatory agents (e.g., ibuprofen), anti-viral agents (e.g., efavirenz, darunavir), chemotherapeutic agents (e.g., dasatinib, imatinib, pazopanib, erlotinib, tofacitinib, paclitaxel, camptothecins), anti-bacterial agents (e.g., rifampicin, bedaquiline), anti-histamines (e.g., cinnarizine) and the like. The purpose of a pharmaceutical composition is to facilitate administration of an active compound to a subject. The terms “active compound”, “active ingredient”, “a therapeutically active agent”, “active agent”, “biologically active agent”, “bioactive agent”, and the like are used herein throughout interchangeably and refer to a compound, which is accountable for biological effect.

The terms “pharmaceutical composition” or “pharmaceutical product” are also to be construed to encompass a cosmetic or cosmeceutical product and a nutrient or a nutraceutical product.

By “biological effect” it is meant to refer to biological, therapeutic or physiological activity in an organism e.g., following administration thereof to a subject.

The terms “physiologically acceptable carrier” and “pharmaceutically acceptable carrier”, which may be interchangeably used, refer to a carrier or a diluent that does not cause significant irritation to an organism and does not abrogate the biological activity and properties of the administered compound.

Herein the term “excipient” refers to an inert substance added to a pharmaceutical composition to further facilitate administration of a drug. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical products for use in accordance with the present invention thus may be formulated in a conventional manner using one or more pharmaceutically acceptable carriers comprising excipients and auxiliaries, which facilitate processing of the compounds into preparations which can be used pharmaceutically. Proper formulation is dependent upon the route of administration chosen. The dosage may vary depending upon the dosage form employed, the route of administration utilized and the patient subpopulation (e.g., adult, pediatric, geriatric). The exact formulation, route of administration and dosage can be chosen by the individual physician in view of the patient's condition (see e.g., Fingl et al, 1975, in “The Pharmacological Basis of Therapeutics”, Ch. 1 p. 1).

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophthalmically, vaginally, rectally, intranasally). Pharmaceutical composition for intravenous or injectable matrix may comprise an effective amount of a biocompatible, biodegradable controlled release material, the material contained in the polymer is selected from: polyanhydrides, lactic acid and glycolic acid copolymers, polyesters (e.g., polylactic acid, and polyglycolic acid), polyethers, polyorthoesters, polyols, proteins, polysaccharides, and any mixture thereof.

Formulations for topical administration may include but are not limited to lotions, ointments, gels, creams, suppositories, drops, liquids, sprays, powders, films and patches. Conventional pharmaceutical carriers, aqueous, powder or oily bases, thickeners and the like may be necessary or desirable.

Compositions or products for oral administration include powders or granules, suspensions or solutions in water or non-aqueous media, sachets, pills, caplets, capsules, tablets or orally-dissolving films. Thickeners, diluents, flavorings, dispersing aids, emulsifiers or binders may be desirable.

Formulations for parenteral administration may include, but are not limited to, sterile solutions or dispersions which may also contain buffers, diluents and other suitable additives. Slow release compositions are envisaged for treatment.

The amount of a composition to be administered will, of course, be dependent on the subject being treated, in the medical condition being treated for, the severity of the affliction, the manner of administration, the judgment of the prescribing physician, etc.

The pharmaceutical composition may further comprise additional pharmaceutically active or inactive agents such as, but not limited to, an anti-bacterial agent, an antioxidant, a buffering agent, a bulking agent, a surfactant, an anti-inflammatory agent, an anti-viral agent, a chemotherapeutic agent and anti-histamine.

According to an embodiment of the present invention, the pharmaceutical composition described herein above is packaged in a packaging material and identified in print, in or on the packaging material, for use in the treatment of a disease or disorder, as described herein.

According to another embodiment of the present invention, the pharmaceutical composition is packaged in a packaging material and identified in print, in or on the packaging material, for use in monitoring a disease or disorder, as described herein.

Products of the present invention may, if desired, be presented in a pack or dispenser device, such as an U.S. Food and Drug Administration (FDA) approved kit, or as a diagnostic kit which may contain one or more unit dosage forms containing the disclosed composition. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the FDA for prescription drugs or of an approved product insert.

As used herein throughout, the terms “encapsulate” and/or “entrap” and their grammatical derivatives and conjugations, as used in the context of the present embodiments, relate to any form of accommodating a substance, herein the active agent, within a closed e.g., structure, herein the self-assembled structure. In some embodiments, the entrapment of the active agent in the self-assembled structure, as in the context of the present embodiments, provides complete integration of the active agent within the structure, such that the entrapped active agents are fully isolated from the surrounding environment as long as the structure is assembled (closed).

As used herein, the terms “encapsulate” and/or “entrap” are meant to encompass cases where the encapsulated entity is solvated, e.g., the encapsulation includes solvent molecules. In cases where the encapsulated entity is surrounded by surface active agents, the encapsulation also includes the surrounding surface-active agents.

The encapsulation, according to the present embodiments, is also meant to include the encapsulation of the solvent in which the encapsulation process takes place and/or the various solutes which are present in the solvent in addition to e.g., the chemical monomers and the active agent.

In cases where the active agent is not soluble under the conditions of the self-assembly process, the active agent can be solubilized by means of surface active molecules that surround the molecules of the active agent, which are encapsulated therewith in the encapsulation process.

As described herein above, the void within a self-assembled core-corona structure wherein the active agent is encapsulated is set by the size of the core, the type of associating groups, and associating mode there between. Hence, the size of the void within the self-assembled structure may be controlled by selecting suitable chemical monomeric units having particular associating groups.

The type of active agent which is suitable for encapsulation within the structure according to the present embodiments depends on several characteristics thereof, such as its size, its solubility in the media in which the self-assembled core-corona structure is formed as well as other chemical compatibility criteria.

In some embodiments, the phrase “active agent”, is referred to a “drug”, that is a compound which exhibits a beneficial pharmacological effect when administered to a subject and hence can be used in the treatment of a condition that benefits from this pharmacological effect.

That is, in some embodiments, the biologically or therapeutically active agent is selected from the group consisting of: prophylactic drugs, anticancer drugs, anti-viral drugs, anti-bacterial drugs, anti-fungal drugs, anti-parasite drugs, or combination of drugs, vitamins or metabolites thereof (e.g., retinoic acid), monoclonal antibody, siRNA, RNA, microRNA, DNA, genes, a vaccine, a plasmid, a labeling agent, a diagnostic agent, proteins e.g., enzymes, antigens, a bisphosphonate, an antibacterial, an anti-viral or an antifungal reagent.

For example, a composition which comprises a therapeutically active agent (e.g., a drug) attached to or encapsulated in a self-assembled core-corona or multi-micellar structure as described herein, can be efficiently utilized for treating a medical condition that is treatable by the active agent.

In some embodiments, the composition may further comprise an additional targeting moiety attached to the self-assembled core-corona or to the multi-micellar structure, which enhances the affinity of the self-assembled structure to the desired bodily site where the therapeutic activity should be exerted (e.g., a specific organ, tissue or cells).

In some embodiments, the composition may further comprise a shuttling moiety (e.g., cell penetrating peptide) attached to the self-assembled core-corona structure, which may enhance the permeability of the self-assembled structure in a specific body barrier or cell membrane.

The term “subject” (alternatively referred to herein as “patient”) as used herein refers to an animal, e.g., a mammal, e.g., a human, who has been the object of treatment, observation or experiment.

The phrase “anticancer agent” or “anticancer drug”, as used herein, describes a therapeutically active agent that directly or indirectly kills cancer cells or directly or indirectly inhibits, stops or reduces the migration and/or proliferation of cancer cells. Anti-cancer agents include those that result in cell death and those that inhibit cell growth, migration, proliferation and/or differentiation. In some embodiments, the anti-cancer agent is selectively toxic against certain types of cancer cells but does not affect or is less effective against normal cells. In some embodiments, the anti-cancer agent is a cytotoxic agent.

The terms “cancer” and “tumor” are used interchangeably herein to describe a class of diseases in which a group of cells display uncontrolled growth (division beyond the normal limits). The term “cancer” encompasses malignant and benign tumors as well as disease conditions evolving from primary or secondary tumors. The term “malignant tumor” describes a tumor which is not self-limited in its growth, is capable of invading into adjacent tissues, and may be capable of spreading to distant tissues (metastasizing). The term “benign tumor” describes a tumor which is non-malignant (i.e. does not grow in an unlimited, aggressive manner, does not invade surrounding tissues, and does not metastasize). The term “primary tumor” describes a tumor that is at the original site where it first arose. The term “secondary tumor” describes a tumor that has spread from its original (primary) site of growth to another site, close to or distant from the primary site.

Non-limiting examples of therapeutically active agents that may be beneficially used in embodiments of the present invention include, without limitation, one or more of an agonist agent, an amino acid agent, an analgesic agent, an antagonist agent, an antibiotic agent, an antibody agent, an antidepressant agent, an antigen agent, an antihistamine agent, an antihypertensive agent, an anti-inflammatory drug, an anti-metabolic agent, an antimicrobial agent, an antioxidant agent, an anti-proliferative drug, an antisense agent, a chemotherapeutic drug, an antiviral, an antiretroviral, a co-factor, a cytokine, a drug, an enzyme, a growth factor, a heparin, a hormone, an immunoglobulin, an inhibitor, a ligand, a nucleic acid, an oligonucleotide, a peptide, a phospholipid, a prostaglandin, a protein, a toxin, a vitamin and any combination thereof.

As used herein, the phrase “labeling agent” refers to a detectable moiety or a probe and includes, for example, chromophores, fluorescent compounds, phosphorescent compounds, heavy metal clusters, and radioactive labeling compounds, as well as any other known detectable moieties.

In any of the methods, uses, compositions, or products, described herein, the products described herein may be utilized in combination with additional therapeutically active agents. Such additional agents include, as non-limiting examples, chemotherapeutic agents, anti-angiogenesis agents, hormones, growth factors, antibiotics, anti-microbial agents, anti-depressants, immunostimulants, and any other agent that may enhance the therapeutic effect of the composition and/or the well-being of the treated subject.

Additional non-limiting examples of active agents include: acetylcholinesterase inhibitors, analgesics and nonsteroidal antiinflammatory agents, anthelminthics, antiacne agents, antianginal agents, antiarrhythmic agents, anti-asthma agents, antibacterial agents, anti-benign prostate hypertrophy agents, immunosuppressants, anticoagulants, antidepressants, antidiabetics, antiemetics, antiepileptics, antifungal agents, antigout agents, antihypertensive agents, antiinflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, antiretroviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, hypnotics, immunosuppressants, keratolytics, lipid regulating agents, leukotriene inhibitors, macrolides, muscle relaxants, neuroleptics, nutritional agents, opiod analgesics, protease inhibitors, sedatives, sex hormones, stimulants, vasodilators, essential fatty acids, non-essential fatty acids, proteins, peptides, sugars, vitamins, nutraceuticals, natural agents, or mixtures thereof.

In some embodiments, the active agent is hydrophobic. Hydrophobic active agents may include agents having many different types of activities.

Non-limiting examples of hydrophobic active agents include: antiproliferatives such as paclitaxel, sirolimus (rapamycin), everolimus, biolimus A9, zotarolimus, tacrolimus, and pimecrolimus and mixtures thereof; analgesics and anti-inflammatory agents such as aloxiprin, auranofin, azapropazone, benorylate, diflunisal, etodolac, fenbufen, fenoprofen calcium, flurbiprofen, ibuprofen, indomethacin, ketoprofen, meclofenamic acid, mefenamic acid, nabumetone, naproxen, oxyphenbutazone, phenylbutazone, piroxicam, sulindac; anti-arrhythmic agents such as amiodarone, disopyramide, flecainide acetate, quinidine sulphate; antibacterial agents such as benethamine penicillin, cinoxacin, ciprofloxacin, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim, rifampicin, bedaquiline; anti-coagulants such as dicoumarol, dipyridamole, nicoumalone, phenindione; antihypertensive agents such as amlodipine, guanethidine, benidipine, darodipine, dilitazem, diazoxide, felodipine, guanabenz acetate, isradipine, minoxidil, nicardipine, nifedipine, nimodipine, phenoxybenzamine, prazosin, reserpine, terazosin; anti-muscarinic agents: atropine, benzhexol, biperiden, ethopropazine, hyoscyamine, mepenzolate bromide, oxyphencylcimine, tropicamide; anti-neoplastic agents and immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, paclitaxel, procarbazine, tamoxifen citrate, testolactone, topotecan, SN38, topotecan, irinotecan, exatecan, lurtotecan, imatinib, nilotinib, dasatinib, bosutinib, ponatinib, erlotinib, pazopanib, tofacitinib, doxorubicin, and combinations thereof; beta-blockers such as acebutolol, alprenolol, atenolol, labetalol, metoprolol, nadolol, oxprenolol, pindolol, propranolol; cardiac inotropic agents such as amrinone, digitoxin, digoxin, enoximone, lanatoside C, medigoxin; corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; lipid regulating agents such as bezafibrate, clofibrate, fenofibrate, gemfibrozil, probucol; nitrates and other anti-anginal agents such as amyl nitrate, glyceryl trinitrate, isosorbide dinitrate, isosorbide mononitrate, pentaerythritol tetranitrate; antiretrovirals such as nevirapine, efavirenz, etravirine, saquinavir, ritonavir, indinavir, lopinavir, darunavir, atazanavir, fosamprenavir, tipranavir, maraviroc, vicriviroc, rilpivirin, and combinations thereof; antiparasitic drugs such as benznidazole, nifurtimox, nitozoxanide, miltefosine and combination thereof. Other hydrophobic active agents include, but are not limited to, active agents for treatment of hypertension (HTN), such as guanethidine.

In some embodiments, the hydrophobic active agents are new chemical entities under in vitro or in vivo preclinical and clinical evaluation.

In some embodiments, the pharmaceutical composition described hereinabove is suitable to be used in the prophylaxis, diagnosis, or therapy in human or veterinary medicine for the release of biologically active cargos such as drugs, enzymes, proteins, genes, or any other agent with prophylactic, diagnostic, or therapeutic properties, or combinations of these properties.

In some embodiments of the invention, the pharmaceutical composition is a topical composition formulated for administration onto the skin (including eyes, scalp, hair and nails) of a subject.

In some embodiments of the invention, the pharmaceutical composition is an injectable composition.

In some embodiments, the composition is characterized as being adhesive to a mucosal tissue.

In some embodiments, the micelles are characterized by mucoadhesiveness e.g., to prolong the residence time of the self-assembled particles in the gastrointestinal track and enhance the absorption and the bioavailability of the encapsulated drug.

In some embodiments, the self-assembled particles are characterized by high physical stability under unfavored conditions (e.g., extreme dilution) and upon interaction with mucosa thereby preserving the drug in the core.

In some embodiments, the self-assembled particles are characterized by a rate-controlling capacity of the drug.

In some embodiments, the self-assembled particles are in the form of micro- or nanogel.

In some embodiments, the pharmaceutical composition is formulated for oral or nasal administration.

In some embodiments, the composition is formulated for mucosal application. As used herein, the term “mucosal application” may refer to absorption of an active agent and the like and is meant to encompass absorption across or through a mucous membrane.

The term “mucoadhesive”, or any grammatical derivative thereof, as used herein refers to the phenomenon where a natural or synthetic substance applied to a mucosal epithelium, adheres, usually creating a new interface, to the mucus layer.

In some embodiments, the pharmaceutical composition is formulated for oral administration. In some embodiments, the pharmaceutical composition is formulated for ophthalmic administration e.g., as eye drops, cream, etc. In some embodiments, the pharmaceutical composition is formulated for intranasal administration. In some embodiments, the pharmaceutical composition is formulated for inhalation. In some embodiments, the pharmaceutical composition is formulated as a pharmaceutically acceptable injectable matrix.

According to another aspect of embodiments of the present invention, there is provided a use of the composition described herein in the manufacture of a medicament for treating a medical condition treatable by the therapeutically active agent. In some embodiments the medicament is formulated for oral administration.

As discussed herein above, a large group of drugs, e.g., chemotherapeutic agents are hydrophobic and exhibit poor solubility in aqueous solution, thus rendering their oral administration problematic. For example, many chemotherapeutic agents are typically administered intravenously. This route of administration is a major source of cost, discomfort and stress to patients, and multiple hospitalizations are required in order to complete the relatively long chemotherapeutic regimen. Thus, the enhancement of water solubility of the chemotherapeutic agent, by encapsulation in the herein described core-corona or multi-micellar structure is especially beneficial and may be utilized for treating e.g., cancer and cancer metastases.

In some embodiments, the composition described hereinabove provides desirable solubility factor.

In some embodiments, the solubility factor is solubility of active agent in the self-assembled particles divided by the intrinsic solubility of the active agent in a polymer-free medium (at 37° C.). A solubility factor of above 1 indicates that more than the amount of active agent soluble in the solvent present.

In some embodiments, the solubility factor is at least 100, least 200, least 300 least 400, least 500, least 600, least 700, least 800, least 900, least 1000, least 1200, least 1300, least 1400, or least 1500.

In some of any of the embodiments of the present invention, the therapeutic agent may also comprise a vasodilator to counteract vasospasm, for example an antispasmodic agent such as papaverine. The therapeutic agent may be a vasoactive agent, generally such as calcium antagonists, or alpha and beta-adrenergic agonists or antagonists. In some of any of the embodiments of the present invention, the therapeutic agent may include a biological adhesive such as medical grade cyanoacrylate adhesive or fibrin glue, the latter being used to, for example, adhere an occluding flap of tissue in a coronary artery to the wall, or for a similar purpose.

In some of any of the embodiments of the present invention, the therapeutic agent may be an antibiotic agent that may be released from the core, optionally in conjunction with a controlled release carrier for persistence, to an infected organ or tissue or any other source of localized infection within the body. Similarly, the therapeutic agent may comprise steroids for the purpose of suppressing inflammation or for other reasons in a bodily site. Exemplary anti-infective agents include, for example, chlorhexidine which is added for improved biocompatibility of articles-of-manufacturing comprising the composition according to some of any of the embodiments of the present invention.

In some embodiments, compositions wherein the associating groups of the domains or monomeric units which form the self-assembled hollow structure are selected are beneficial for use in drug delivery. One exemplary use of such a composition wherein specific drug delivery is crucial is a composition for gene therapy. Such a composition can include a self-assembled hollow structure as presented herein and a combination of active agents attached to and/or encapsulated in a self-assembled particle. In order to design an effective tool for local gene therapy, a chemical self-assembled core-corona or multi-micellar structure may have the following components: a nucleic acid construct, an antisense or any other agent useful in gene therapy, for effecting the desired therapeutic effect within a cell, being attached to the self-assembled core-corona structure or being encapsulated in a self-assembled core-corona or multi-micellar structure, that can be disassembled under physiological conditions (e.g., being biocleavable by cellular components); and an additional targeting agent attached to the self-assembled core-corona structure, selected to have an affinity to the desired location and optionally having a capacity for internalization into the cells at the desired location. Once reaching its designed target, the self-assembled core-corona or multi-micellar structure is internalized into the cells and once inside the cell, the therapeutic agent is released upon interaction with cellular components that e.g., cleave either the interactions within the disclosed polymeric structure. Thus, the therapeutic agent is delivered to its final target and can exert its therapeutic activity. In some embodiments, the composition comprises a self-assembled core-corona or multi-micellar structure as described herein and an anti-cancer drug attached thereto or encapsulated therein.

In some embodiments, the therapeutic agent is released from the core, if the concentration of the self-assembled core-corona or multi-micellar structures is below the CMC. Apart from having unique, drug delivery attributes, as detailed herein, the self-assembled core-corona or multi-micellar structures may include an active agent such as a labeling agent attached to or encapsulated therein. The encapsulation of an additional active agent to a self-assembled core-corona or multi-micellar structure having a labeling moiety attached thereto or encapsulated therein may afford an efficient imaging probe. When the additional active agent is a targeting moiety, the encapsulation thereof to self-assembled core-corona or multi-micellar structures having a labeling agent attached thereto or encapsulated therein, can assist in the location, diagnosis and targeting of specific loci in a host. When the additional active agent is a therapeutic agent, the encapsulation thereof to self-assembled core-corona or multi-micellar structures having a labeling agent (e.g., indocyanine green) attached thereto or encapsulated therein, can assist in monitoring the distribution thereof in the host, thereby monitoring medical conditions as described herein below. The use of the self-assembled core-corona or multi-micellar structure as a delivery vehicle depends largely on its capacity to penetrate at least some physiological barriers and biologic degradation. To this effect, the chemical self-assembled core-corona or multi-micellar structure can be functionalized so as to alter its surface for higher biocompatibility. Such alteration can improve the bioavailability of any other active agents attached to and/or encapsulated in the self-assembled hollow structure. Exemplary surface-active agents that can provide such bioavailability include certain polymers, which are known to exert the desired surface alteration to organic and non-organic entities.

In some embodiments, the self-assembled structure disclosed herein or at least a part thereof has mucus adhesive properties.

In some embodiments, there is provided herein a method for extending the release period in a physiological environment (e.g., blood) of at least one active agent, the method comprising encapsulating at least one active agent in the core-corona or multi-micellar structure disclosed herein. In some embodiments, active agent is slowly released. In some embodiments, active agent is released in a controlled manner. In some embodiments, active agent is identified as being unstable in the physiological environment. In this context, the term “controlled manner” indicates that the drug is released substantially constantly, or in accordance with a pre-defined rate to e.g., a target cell. Herein, the term “constantly” may refer to a time duration of about e.g., 30 min, 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days, including any value there between.

In some embodiments, the disclosed composition is enabled to encapsulate therein active substance via a variety of interactions depending on the intended use of the desired release characteristics of the active substance, the desired surface properties of the object and many more.

In some embodiments, the composition disclosed herein is designed as uniform and adherent coating and may further be designed to controllably release active substances that are encapsulated therein.

Method of Treatments

According to some embodiments, there is provided a method of monitoring medical conditions such as, but are not limited to, cancer and infections (e.g., viral infection, parasitic infection, fungal infection or bacterial infection).

It is to note that herein, by targeting a therapeutically active agent via the methodologies described herein, the toxicity of the therapeutically active agent is substantially reduced both systemically and locally in body sites where the active agent is not expected to exert its activity. Consequently, besides the use of the amphiphilic polymers described herein in a clinically evident disease, optionally in combination with other drugs, these amphiphilic polymers may potentially be used as a long term-prophylactic for individuals who are at risk for relapse due to residual dormant cancers.

As further detailed hereinabove, the term “cancer” or “cancer cells” describes a group of cells which display uncontrolled growth (division beyond the normal limits).

As described hereinabove, cancers treatable by the compositions described herein include, but are not limited to, solid, including carcinomas, and non-solid, including hematologic malignancies.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating”, or any grammatical derivative thereof, is meant to refer to abrogating, substantially inhibiting, slowing or reversing the progression of a condition, substantially ameliorating clinical or aesthetical symptoms of a condition or substantially preventing the appearance of clinical or aesthetical symptoms of a condition.

It is understood that the composition of the present invention may be administered in conjunction with other drugs, including other antiviral and/or anti-cancer drugs.

As further described hereinabove, the composition of the invention may comprise a labeling agent. Composition comprising a labeling agent may be used in suitable imaging techniques.

Suitable imaging techniques include but are not limited to positron emission tomography (PET), computed tomography (CT), gamma-scintigraphy, magnetic resonance imaging (MRI), functional magnetic resonance imaging (fMRI), magnetoencephalography (MEG), single photon emission computerized tomography (SPECT), computed axial tomography (CAT) scans, ultrasound, fluoroscopy and conventional X-ray imaging, or any combination there between.

The choice of an appropriate imaging technique depends on the nature of the labeling agent, and is within the skill in the art. For example, if the labeling agent comprises Gd ions, then the appropriate imaging technique is MRI; if the labeling agent comprises gamma-emitting radionuclides, an appropriate imaging technique is gamma-scintigraphy; if the labeling agent comprises an ultrasound agent, ultrasound is the appropriate imaging technique; if the labeling agent comprise a near infrared (NIR) dye, NIR is the appropriate technique; etc.

The Process

According to another aspect of embodiments of the present invention, there is provided a process of preparing the amphiphilic block or graft polymer being in form of closed self-assembled core-corona or multi-micellar structure described herein, the closed hollow and self-assembled structure further comprising one or more active agents, the process comprising the steps of:
grafting or copolymerizing a hydrophobic polymeric backbone to a hydrophilic polymeric backbone, thereby forming an amphiphilic block or graft polymer configured to form a self-assembled structure comprising a hydrophobic core and a hydrophilic corona;
mixing the amphiphilic block copolymer with a solvent at a concentration above a predefined minimal concentration, thereby forming a solution;
adding an active agent to the solution e.g., as a solid or in an organic solvent that is water-miscible, thereby encapsulating the active agent within the hydrophobic core.

In some embodiments, the copolymerization or grafting is performed as described herein below (e.g. in the Examples section).

In some embodiments, mixing the amphiphilic block copolymer with one or more solvents results in a solution. In some embodiments, mixing the amphiphilic block copolymer with one or more solvents results in a dispersion. In some embodiments, the solvent is an aqueous solvent (e.g. water or an aqueous salt solution) or an organic water miscible solvent (e.g. a short-chain alcohol, DMSO, DMF). In some embodiments, a solution or a dispersion comprising the amphiphilic block copolymer is an aqueous solution or dispersion. In some embodiments, the process further comprises eliminating one or more solvents by evaporation or dialysis to produce a dry powder.

In some embodiments, the process further comprises a step of reconstitution of the dry powder. In some embodiments, the dry powder is further reconstituted in an aqueous solution.

In some embodiments, the solvent is a described herein above.

In some embodiments, the predefined minimal concentration is critical micelle concentration (CMC).

In some embodiments, the process further comprises a step of heating an aqueous solution containing the amphiphilic block polymer to a temperature that ranges from about 30° C. to about 50° C. (e.g., 37° C.) prior to the step of adding the active agent to the solution. In some embodiments, the process further comprises incubation at a temperature ranging from 20 to 50° C. In some embodiments, the incubation is for at least one hour. In some embodiments, the incubation results in formation of self-assembled structures. In some embodiments, self-assembled structures are formed prior to the step of adding the active agent.

In some embodiments, the process further comprises a step of cooling the solution to a temperature lower than 30° C., thereby stabilizing the amphiphilic block polymer.

In some embodiments, the process further comprises a step of diluting the dispersion to a final concentration below the predefined concentration, following the step of adding an active agent to the dispersion thereby stabilizing the amphiphilic block polymer.

In some embodiments, the process comprises: (i) mixing both the active agent and the amphiphilic block copolymer, to obtain a mixture; (ii) dissolving the mixture in a solvent, to obtain a solution or a dispersion comprising the amphiphilic block copolymer at a concentration above a predefined minimal concentration; (iii) optionally drying the solution or a dispersion, to produce a dry powder.

Herein, the step of polymerizing the hydrophobic component may be performed by any polymerization method described hereinabove, for example by subjecting the corresponding monomers to conditions that allow them to associate there between via their associating groups.

Herein, the active agent may be any active agent described hereinabove.

In some embodiments the process described herein, is such wherein the concentration of the active agent in the solution and the concentration of the amphiphilic block polymer in the aqueous solution are selected so as to obtain a pre-determined molar ratio of the active agent to the amphiphilic block polymer.

The construction of the desired amphiphilic block polymer, comprising the encapsulated active agent may be verified by techniques known in the art.

Examples of such techniques include, zeta-potential (Z-potential) measurements, DLS, electron microscopy, etc.

General

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As used herein, the term “alkyl” describes an aliphatic hydrocarbon including straight chain and branched chain groups. The alkyl group has 1 to 100 carbon atoms, and more preferably 1-50 carbon atoms. Whenever a numerical range; e.g., “1-100”, is stated herein, it implies that the group, in this case the alkyl group, may contain 1 carbon atom, 2 carbon atoms, 23 carbon atoms, etc., up to and including 100 carbon atoms. In the context of the present invention, a “long alkyl” or “high alkyl” is an alkyl having at least 10, or at least 15 or at least 20 carbon atoms in its main chain (the longest path of continuous covalently attached atoms), and may include, for example, 10-100, or 15-100 or 20-100 or 21-100, or 21-50 carbon atoms. A “short alkyl” or “low alkyl” has 10 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” or “cycloalkane” describes an all-carbon monocyclic or fused ring (i.e., rings that share an adjacent pair of carbon atoms) group where one or more of the rings does not have a completely conjugated pi-electron system. The cycloalkyl group may be substituted or unsubstituted, as indicated herein.

The term “aryl” or “aromatic” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂—R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a group.

As used herein, the term “azide” refers to a —N₃ group.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples which, together with the above descriptions, illustrate the invention in a non-limiting fashion.

### Materials

The synthesis of the different copolymers is presented in the following examples.

In exemplary procedures, locust bean gum (LBG) galactomannan (GM) (Sigma-Aldrich, St. Louis, Mo.), cerium ammonium nitrate (CAN, STREM Chemicals, Newburyport, Mass., USA), tetramethylethylenediamine (TEMED, Alfa Aesar, Heysham, UK), hydroxyquinone (HQ, Merck, Hohenbrunn, Germany), nitric acid 70% (Bio-Lab, Jerusalem, Israel) were used as received. Methyl methacrylate (MMA, Alfa Aesar) was distilled under vacuum before use to remove inhibitor. All the other solvents were of analytical or spectroscopic grade and purchased from Bio-Lab or Gadot (Netanya, Israel) and used without further purification. To obtain low molecular weight, low viscosity and higher water solubility GM, hydrolysed GM (hGM) was produced. For this, GM was hydrolysed using trifluoroacetic acid (TFA, Sigma-Aldrich). Briefly, GM (1 g) was dispersed in TFA 1M (40 mL) and heated at 80° C. for 1 h under magnetic stirring (100 rpm). Then, the mixture was dialyzed (regenerated cellulose dialysis membranes; molecular weight cut off of 3500 g/mol; Spectra/Por® 3 nominal flat width of 45 mm, diameter of 29 mm and volume/length ratio of 6.4 mL/cm; Spectrum Laboratories, Inc., Rancho Dominguez, Calif., USA) against distilled water for 24 h and filtered (Whatman® Filter Paper, Grade 91, 10 μm, Piscataway, N.J., USA). Finally, hGM was freeze-dried (48-72 h, Labconco Free Zone 4.5 plus L Benchtop Freeze Dry System, Labconco, Kansas City, Mo., USA) and stored at −20° C. until use.

### Example 1

### Synthesis and Characterization of GM-g-PMMA Copolymers

In exemplary procedures, galactomannan (GM, 0.55 g) was dissolved in 100 mL of distilled water, heated to 85° C. and magnetically stirred (1 h), the solution was cooled down and filtered by filter paper number 1 (11 μm) and degassed by sonication (30 min). TEMED solution (0.18 mL in 50 mL of degassed water) was added to the GM solution and purged with N₂ (30 min) at RT. The GM solution was then heated to 35° C., MMA (amount of 0.2, 0.1, 0.066 or 0.05 g) was directly poured into the solution.

Finally, a CAN solution (0.66 g in 2 mL of degassed water) was added and the reaction proceeded under N₂ atmosphere (3 h, 35° C.). The polymerization was finished by the addition of HQ (0.132 g). Reaction crudes were dialyzed against distilled water (regenerated cellulose dialysis membranes; molecular weight cut off 3500 Da; nominal flat width of 46 mm, diameter of 29.3 mm and volume/length ratio of 6.74 mL/cm 28 μm, wall thickness; Cellu. Sep, Membrane Filtration Compositions, Inc., Seguin, Tex., USA) in 100-fold of volume of the dialyzed solution and freeze-dried (72-96 h) and were stored at 4° C. until use. Reaction yields ranged between 60% and 81%.

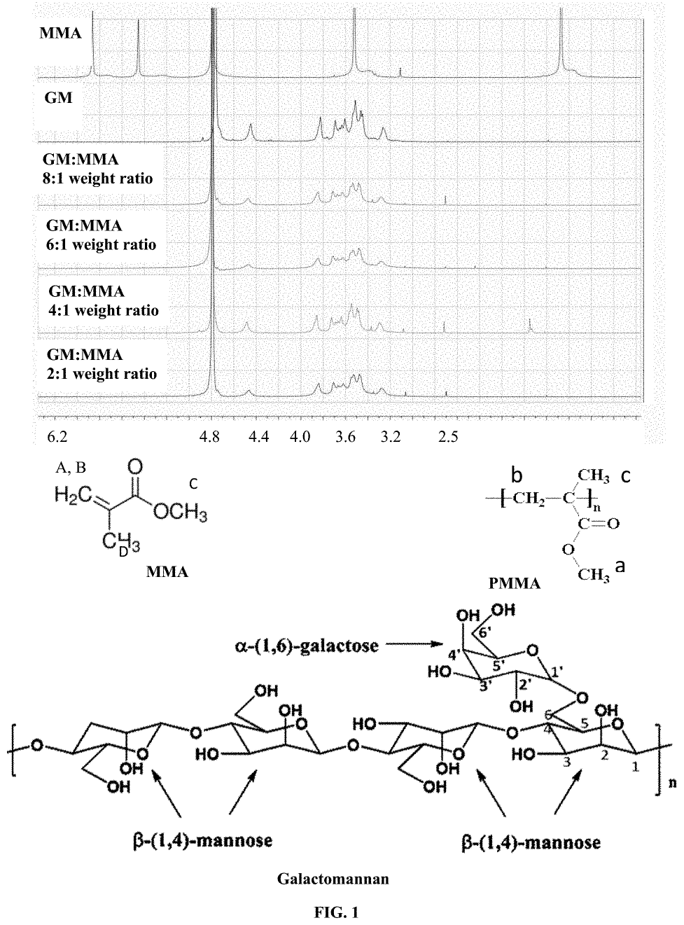

Copolymers were characterized by proton nuclear magnetic resonance (¹H-NMR, 400-MHz Bruker® Avance III High Resolution spectrometer, Bruker BioSpin GmbH, Rheinstetten, Germany), and MestReNova software using 5% w/v solutions in deuterium oxide (D₂O, Sigma-Aldrich) solutions. Chemical shifts are reported in ppm using the signal of H₂O (4.79 ppm) as internal standard. ¹H-NMR spectra of the copolymers were similar to the pure GM spectrum with the appearance of peak C of PMMA at 3.50-3.40 ppm. Peaks of PMMA at 1.00-0.60 ppm were not apparent due to the formation of self-assembled particles in D₂O. At the same time, peaks of the double bonds of MMA (monomer) at 5-6 ppm disappeared, evidencing the successful grafting (FIG. 1).

### Example 2

### Micellization of GM-g-PMMA Copolymers

The aggregation of GM-g-PMMA copolymers was measured by dynamic light scattering (DLS, Zetasizer Nano-ZS, Malvern Instruments, Malvern, UK). For this, stock aqueous solutions of the copolymers (0.1% w/v in water) with reaction GM:MMA weight ratios of 2:1 and 8:1 were prepared by direct dissolution, diluted (0.0001-0.1% w/v) in water and stabilized at RT and at 37° C. overnight. Then, the intensity of the scattered light (DCR) expressed in kilo counts per second (kcps) was measured by DLS and plotted as a function of the copolymer concentration (% w/v).

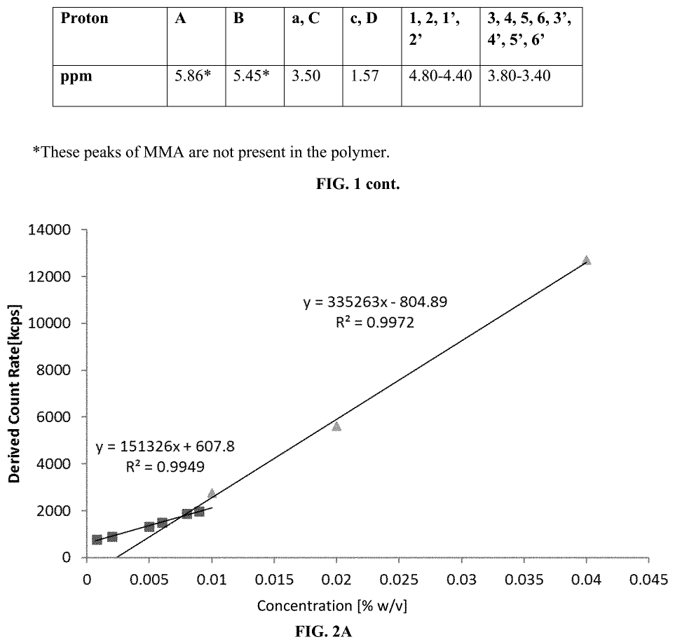

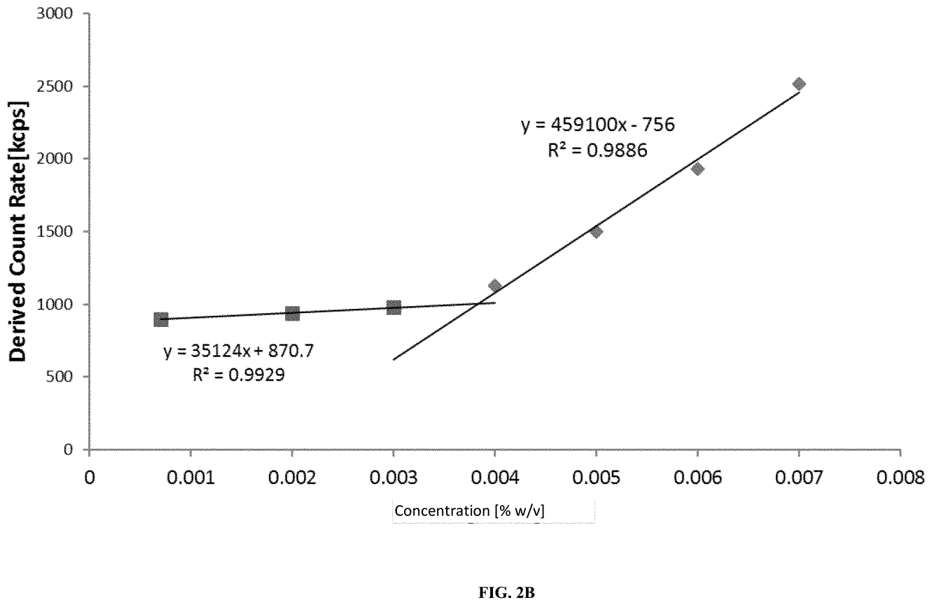

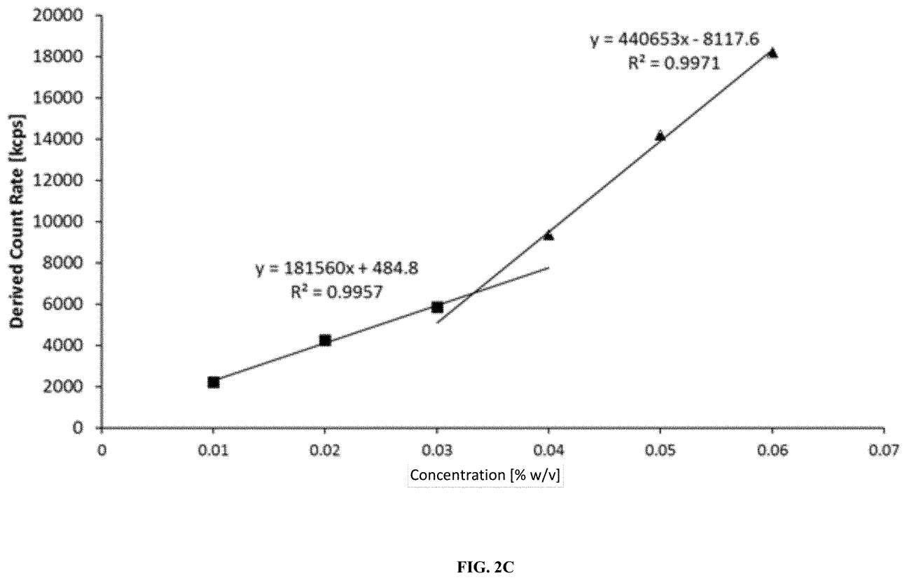

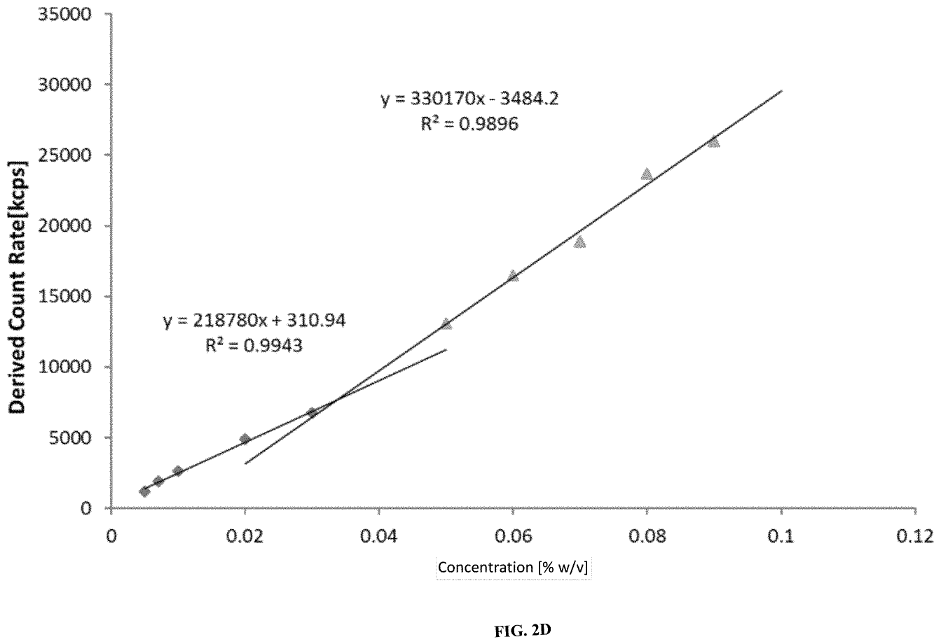

Measurements were carried out at a scattering angle of 173° to the incident beam and data were analyzed using CONTIN algorithms (Malvern Instruments). Data for each single specimen was the result of at least six runs. The micellization was observed as a sharp increase in the scattering intensity and the intersection between the two straight lines corresponded to the critical micellar concentration (CMC). CMC data are expressed in % w/v (FIG. 2). CMC values were between 3.8×10⁻³ and 34×10⁻³% w/v, as summarized below in Table 1.

TABLE 1

| | |
|---|---|
| GM:MMA | |
| weight ratio | CMC (% w/v) |

| in the reaction | 25° C. | 37° C. |
|---|---|---|
| 8:1 | 0.034 | 0.033 |
| 2:1 | 0.004 | 0.008 |

In addition, higher amounts of grafted MMA led to an increase of the hydrophobicity and decrease of the CMC. The temperature had negligible effect on the CMC.

The hydrodynamic diameter (Dh), the size distribution (polydispersity index, PDI) of 0.1% w/v GM-g-PMMA particles were measured by DLS and summarized below in Table 2.

TABLE 2

| GM:MMA | | | | | | |
|---|---|---|---|---|---|---|
| weight ratio | Dₕ ± S.D. (nm) (% Intensity) | | | | PDI ± S.D. | |
| in the reaction | 25° C. | | 37° C. | | 25° C. | 37° C. |
| 8:1 | 2283 ± 1016 | (67%) | 1444 ± 125 | (100%) | 0.61 ± 0.09 | 0.18 ± 0.10 |
| | 540 ± 98 | (32%) | | | | |
| 6:1 | 1495 ± 287 | (100%) | 854 ± 70 | (100%) | 0.57 ± 0.12 | 0.52 ± 0.10 |
| 4:1 | 823 ± 116 | (100%) | 760 ± 59 | (100%) | 0.51 ± 0.05 | 0.51 ± 0.02 |
| 2:1 | 1007 ± 168 | (100%) | 773 ± 155 | (100%) | 0.53 ± 0.03 | 0.43 ± 0.04 |

### Example 3

### Synthesis and Characterization of hGM-g-PMMA Copolymers

In exemplary procedures, hydrolyzed galactomannan (hGM, 0.40 g) was dissolved in 150 mL of distilled water and degassed by sonication (30 min). TEMED solution (0.18 mL in 50 mL of degassed water) was added to the hGM solution and purged with N₂ (30 min) at RT. The hGM solution was then heated to 35° C., MMA (amount of 0.2, 0.1, 0.066 or 0.05 g) was directly poured into the solution. Finally, a CAN solution (0.66 g in 2 mL of degassed water) was added and the reaction proceeded under N₂ atmosphere (3 h, 35° C.). The polymerization was finished by the addition of HQ (0.132 g). Reaction crudes were dialyzed against distilled water (regenerated cellulose dialysis membranes; molecular weight cut off 3500 Da; nominal flat width of 46 mm, diameter of 29.3 mm and volume/length ratio of 6.74 mL/cm 28 μm, wall thickness) in 100-fold of volume of the dialyzed solution and freeze-dried (72-96 h) and were stored at 4° C. until use. Reaction yields ranged between 73% and 87%.

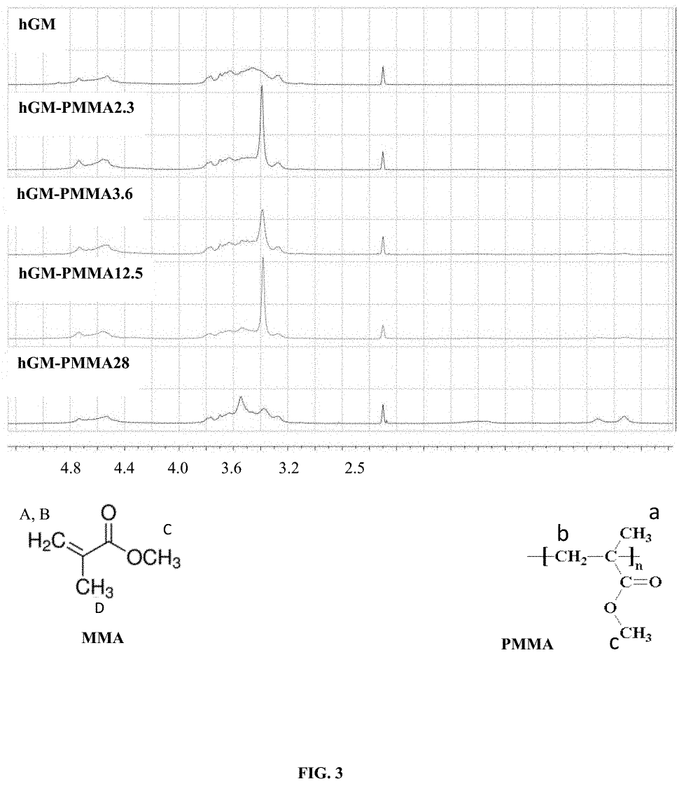

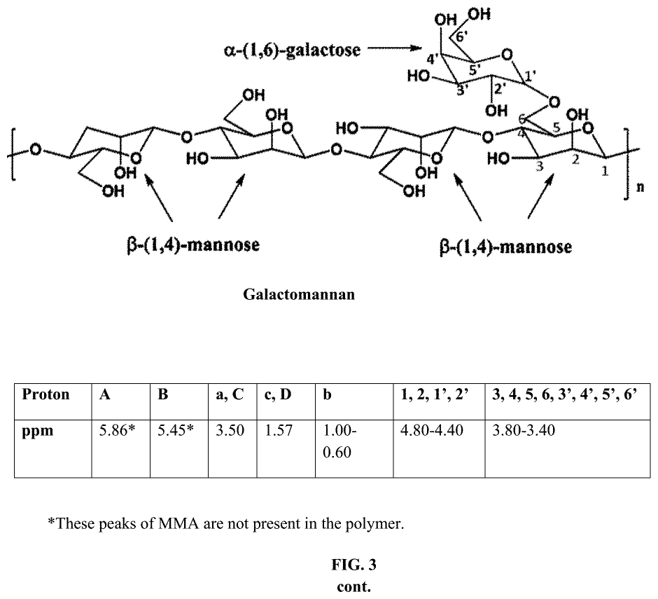

Copolymers were characterized by proton nuclear magnetic resonance (¹H-NMR, 400-MHz Bruker® Avance III High Resolution spectrometer) and MestReNova software using 5% w/v solutions in dimethyl sulfoxide-d6 (DMSO-d6, Sigma-Aldrich) solutions. Chemical shifts are reported in ppm using the signal of DMSO (2.50 ppm) as internal standard. ¹H-NMR spectra of the copolymers were similar to the pure hGM spectrum with the appearance of peaks of PMMA at 3.50-3.40 and 1.00-0.60 ppm. At the same time, peaks of the double bonds of MMA (monomer) at 5-6 ppm disappeared, evidencing the successful grafting (FIG. 3).

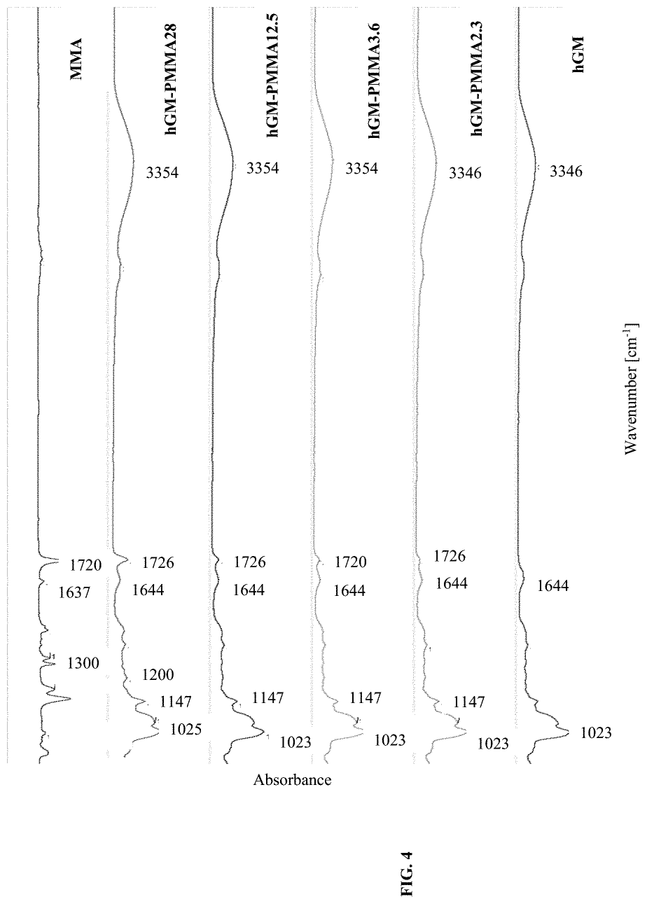

Fourier-Transform infrared spectroscopy (FTIR, Equinox 55 spectrometer, Bruker optics Inc., Ettlingen, Germany; using KBr windows) and differential scanning calorimetry (DSC, DSC 2 STAR′ system simultaneous thermal analyzer using the STAR′ Software V13 (Metter-Toledo; Schwerzenbach, Switzerland, with intracooler Huber TC100 under dry N₂ atmosphere). FTIR spectrum of hGM shows bands of α-D-galactopyranose and β-D-mannopyranose rings at 811 and 871 cm⁻¹. Bands at 1123 and 1120 cm⁻¹ are assigned to primary alcohol bending, broad bands at 2800-3000 cm¹ are attributed to C—H stretching and broad bands at 3100-3600 cm¹ to primary alcohol stretching. All hG-g-PMMA polymers showed the characteristic band of carbonyl 1720 cm⁻¹, confirming the successful graft polymerization. The absence of a C═C band at 1637 cm⁻¹ indicates that unreacted MMA residues are not present (FIG. 4).

To determine the experimental hGM:PMMA ratio in the different polymers, a calibration curve of hGM:MMA physical mixture solutions in D₂O by ¹H-NMR was built (FIG. 5).

The percent of grafting (% Grafting) was calculated according to the following Equation:

% Grafting=% PMMA/(100−% PMMA)×100

The hydrophilic-lipophilic balance (HLB) was estimated by a modification the Griffin's method according to the following Equation:

HLB=20×(Mₕ/M)

where Mₕ is the weight content of the hydrophilic component (hGM) and M is the total weight of polymer.

In general, the polymers are named as hGM-PMMAX, where X is the weight percent (% w/w) of PMMA in the molecule, as determined by ¹H-NMR. The final hGM:PMMA weight ratios and the corresponding % Grafting and HLB values are summarized below in Table 3.

TABLE 3

| | GH:MMA feeding | GH:PMMA | | | |
|---|---|---|---|---|---|
| | weight ratio | weight ratio | Total PMMA | | |
| Polymer | in reaction | in polymer | content (% w/w) | % Grafting | HLB |
| hGM- | 2:1 | 2.5:1 | 28 | 38.9 | 14.4 |
| PMMA28 | | | | | |
| hGM- | 4:1 | 7:1 | 12.5 | 14.3 | 17.5 |
| PMMA12.5 | | | | | |
| hGM- | 6:1 | 27:1 | 3.6 | 3.7 | 19.3 |
| PMMA3.6 | | | | | |
| hGM- | 8:1 | 43:1 | 2.3 | 2.4 | 19.5 |
| PMMA2.3 | | | | | |

### Example 4

### Micellization of hGM-g-PMMA Copolymers

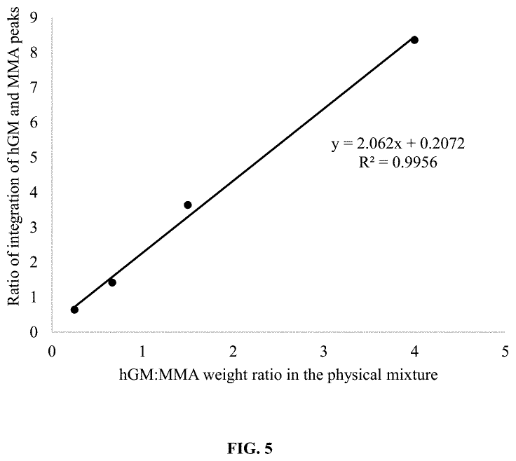

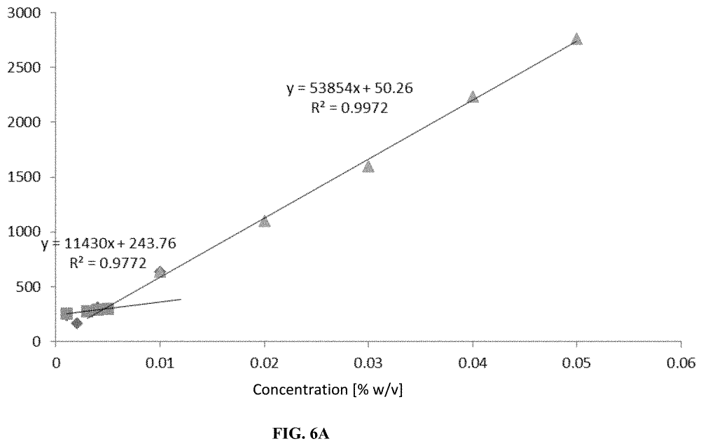

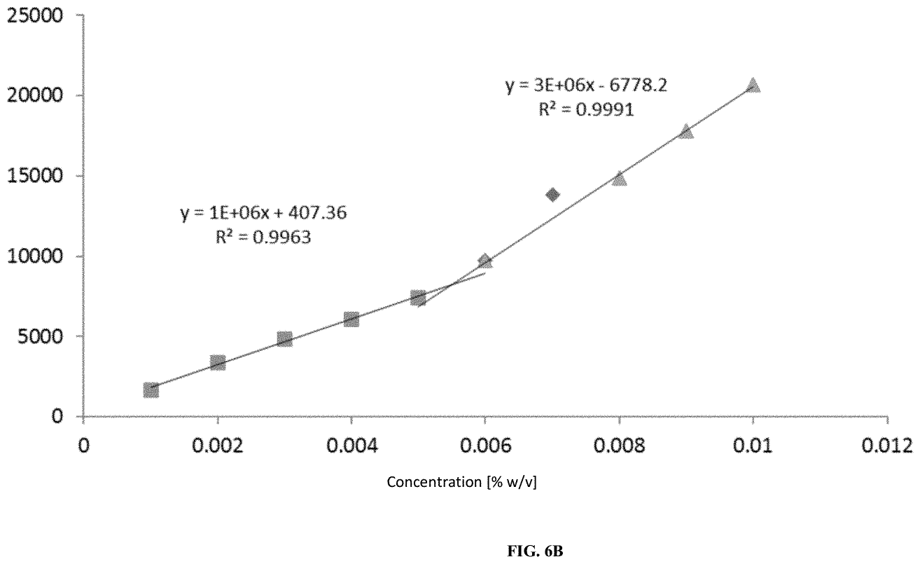

The aggregation of GM-g-PMMA copolymers was measured by dynamic light scattering (DLS). For this, stock aqueous solutions of polymers hGM-PMMA 2.3 and hGM-PMMA28 (0.1% w/v in water) were prepared by direct dissolution, diluted (0.0001-0.1% w/v) in water and stabilized at RT overnight. Then, the intensity of the scattered light (DCR) expressed in kilo counts per second (kcps) was measured by DLS and plotted as a function of the copolymer concentration (% w/v). Measurements were carried out at a scattering angle of 173° to the incident beam and data were analyzed using CONTIN algorithms (Malvern Instruments). Data for each single specimen was the result of at least six runs. The micellization was observed as a sharp increase in the scattering intensity and the intersection between the two straight lines corresponded to the critical micellar concentration (CMC). CMC data are expressed in % w/v (FIG. 6). CMC values at 25° C. were 4.5×10⁻³ and 2.9×10⁻³% w/v for hGM-PMMA2.3 and hGM-PMMA28, respectively, as summarized below in Table 4.

TABLE 4

| Polymer | CMC (% w/v) |
|---|---|
| hGM-PMMA2.3 | 0.005 |
| hGM-PMMA28 | 0.003 |

CMC values at 25° C. and at 37° C. for hGM-PMMA28 are summarized in Table 4A.

TABLE 4A

CMC (% w/v)

| Copolymer | 25° C. | 37° C. |
|---|---|---|
| GalM-g-PMMA | 0.0038 | 0.076 |

Higher amounts of grafted MMA led to an increase of the hydrophobicity and decrease of the CMC. The temperature had negligible effect on the CMC.

The hydrodynamic diameter (Dh), the size distribution (polydispersity index, PDI), and Zeta potential of 0.1% w/v hGM-g-PMMA particles were measured by DLS at 25° C. and summarized below in Table 5. Additionally, Table 5 provides a Dh value for hGM-g-PMMA particles measured by Nanoparticle Tracking Analysis (NTA).

TABLE 5

| | | DLS analysis | | | | | NTA analysis |
|---|---|---|---|---|---|---|---|
| | T | Dₕ (nm) ± S.D. | | | Number Mean | Z-Potential | Dₕ (nm) ± S.D. |
| Copolymer | [° C.] | (Relative intensity %) | | PDI | (nm) ± S.D. | (mV) | (Relative intensity %) |
| GalM-g-PMMA | 25 | 145 ± 8 | (100) | 0.4 ± 0.12 | 96 ± 7 | −6.6 | 102 ± 3 (100) |
| | 37 | 141 ± 3 | (100) | 0.1 ± 0.01 | 84 ± 27 | −5.1 | |
| GalM-g-PMMA | 37 | 129 | (100) | 0.3 | 68 ± 15 | −3.5 | |
| in 90% BSA | | | | | | | |

### Example 5

### In Vitro Cell Compatibility of hGM-PMMA28 Particles

Since these amphiphilic particles are envisioned for administration by different routes and for the targeting of cancer cells, in exemplary embodiments, their cell compatibility was evaluated in the rhabdomyosarcoma cell line Rh30. Rh30 cells were cultured in RPMI-1640 medium (RPMI, Life Technologies Corp., Carlsbad, Calif., USA) supplemented with L-glutamine and NaHCO₃, 10% heat-inactivated fetal bovine serum (FBS, Sigma-Aldrich) and 5 mL of penicillin/streptomycin (commercial mixture of 100 U/mL de penicillin+100 μg/mL streptomycin per 500 mL medium, Sigma-Aldrich), maintained at 37° C. in a humidified 5% CO₂ atmosphere. Rh30 cells were split every 2-3 days, trypsinized (trypsin-EDTA 0.25% w/v, Sigma-Aldrich) and the number of living cells quantified using the trypan blue (0.4% w/v, Sigma-Aldrich) exclusion assay and a Neubauer chamber. For cell compatibility, cells were cultured in 96-well plates (7.5×10³ cells/well) and allowed to attach to the flask bottom for 72-96 h. Then, the culture medium was replaced by 180 μL fresh medium and 20 μL of pristine T1107, T1107-Glu, T1107-LA and T1107-Glu-LA solution in PBS to a final copolymer concentration of 0.5% w/v; PM dispersions were sterilized by filtration through sterile 0.22 μm syringe filters (Merck Millipore Ltd.) before use. After 4 and 24 h incubation, the medium was replaced by 100 μL of fresh medium and 25 μL of sterile 3-(4,5-dimethylthiazol-2-yl)-2,5-diphenyltetrazolium bromide solution (MTT, 5 mg/mL, Sigma-Aldrich), incubated for 4 h (37° C., 5% CO₂), formazan crystals dissolved in DMSO (100 μL) and the absorbance measured at 530 nm (with reference to the absorbance at 670 nm) in a UV-Visible microplate reader (Multiskan GO, Thermo Scientific, Waltham, Mass., USA). The percentage of live cells was estimated with respect to a control treated only with culture medium and that was considered 100% viability.

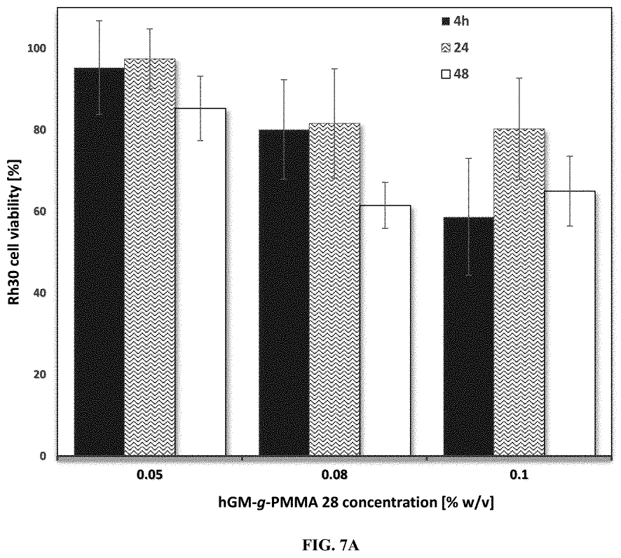

FIGS. 7A and 7B summarize the viability of Rh30 cells and RAW 264.7 cells respectively. The cells were incubated with hGM-PMMA particles. Cells incubated with medium were used as control, that was considered 100% viability.

### Example 6

### In Vitro Cell Uptake of hGM-PMMA28 Particles

The polymeric particles are envisioned for active cell uptake by receptors and/or transporters in cell membranes. In exemplary embodiments, the uptake was assessed in the Rh30 cell line that expresses glucose transporter-1 (GLUT-1). For this, hGM-PMMA28 was fluorescently-labeled with the fluorescent dye fluorescein 5(6)-isothiocyanate (FITC, Sigma-Aldrich). Briefly, 0.1 g of polymer was dissolved in 2 mL of dry DMF (final concentration 20% w/v, Sigma-Aldrich) under magnetic stirring. Then, 10 mg of FITC was dissolved in 200 μL of dry DMF, added to the copolymer solution. The reaction was allowed to proceed for 16 h at 32° C. under magnetic stirring protected from light. The resulting solution was diluted in 10 mL of water, dialyzed against distilled water frequent water exchange in order to eliminate unconjugated FITC until no fluorescence was detected in the dialysis medium and freeze-dried. Then, the fluorescently labelled derivative was mixed with unlabeled hGM-PMMA28 in a unlabeled:labeled ratio of 70:30 to obtain fluorescently-labeled particles to a final polymer concentration of 1% w/v at 37° C. Then, particle dispersions were sterilized by filtration with sterile 0.22 μm syringe filter and diluted in PBS to the final concentration for the assay. For confocal microscopic studies, Rh 30 cells were grown on glass coverslips, in 24 well plates (5×10⁴ cells/well).

The cells were incubated with 500 μl of 0.1% hGM-PMMA28 particles dispersed in DMEM-F12-10% serum containing medium for 4 and 24 h at 37° C. in a CO₂ incubator. Control cells were incubated with DMEM-10% serum containing medium without particles. The treated cells were washed three times with phosphate buffered saline (PBS, pH 7.4) to remove free-floating particles and fixed for 20 min with paraformaldehyde (4% w/v) at RT. For actin staining, fixed cells were permeabilized with 0.1% Triton-X for 5 min at RT. Non-specific binding was blocked by treating with 2% bovine serum albumin in PBS for 30 min. Phalloidin-Atto 647N (red fluorescence, 65906, Sigma-Aldrich) was diluted with PBS and incubated for 15-20 minutes at the RT. After washing with PBS, the stained preparations were mounted with Fluoroshield Mounting Medium with DAPI (blue fluorescence, staining of the nucleus, ab104139) and were left to dry in dark for 24 h and then, visualized with a confocal microscope Zeiss LSM 710 (Heidelberg, Germany).

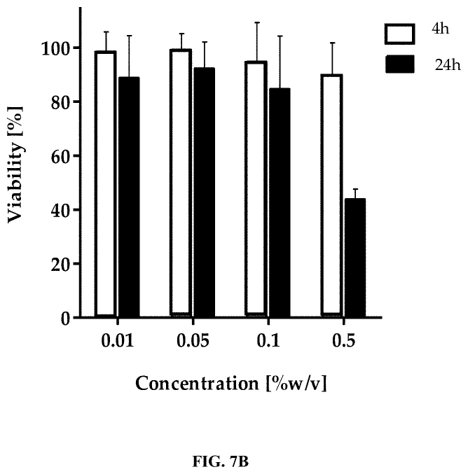

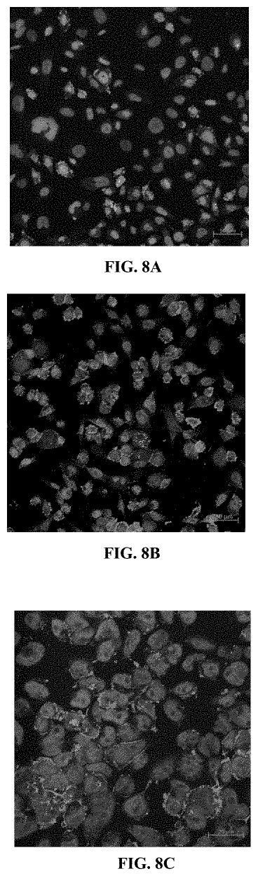

The internalization of hGM-PMMA28 particles by Rh30 cells is presented in FIG. 8.

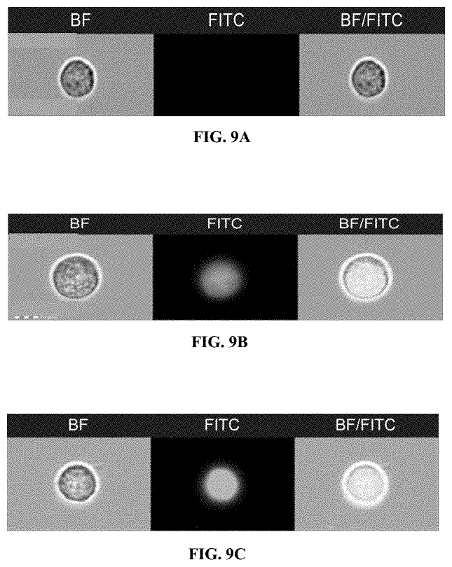

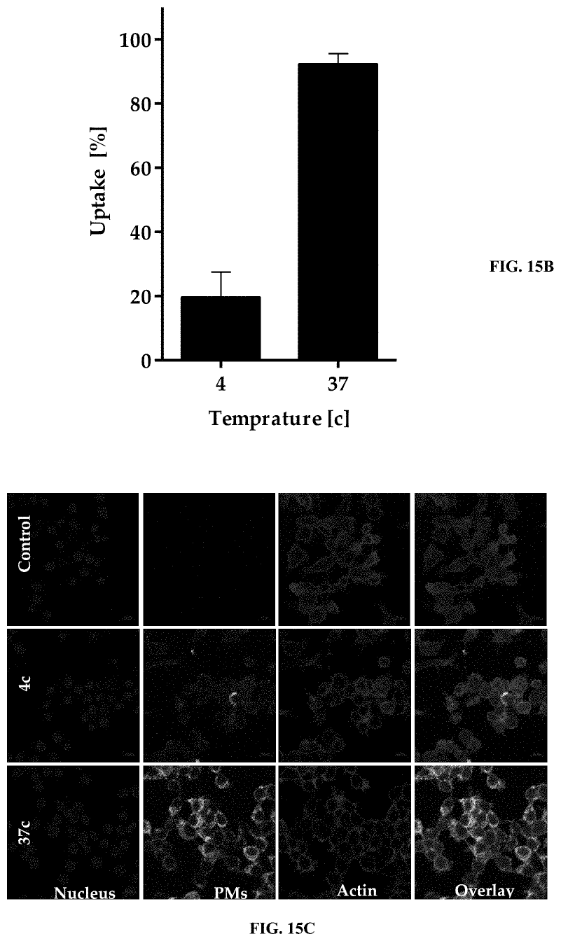

To reveal the involvement of an energy-dependent uptake mechanism, cells were incubated under different temperature conditions: at 4° C. for 1 h (FIG. 8A) where negligible uptake was observed, and at 37° C. for 4 h (FIG. 8C) where an increasing uptake was observed. Then, visualization of the cells was carried out as depicted before. As shown by FIG. 8C, hGM-PMMA28 particles don't exhibit nuclear localization. Additionally, no colocalization of actin fluorescence and particles fluorescence was observed. Cells incubated with fluorescently-labeled hGM-PMMA28 particles at 4° C. did not show fluorescence (FIG. 9A). Conversely, at 37° C., 100% of the cells were stained after 4 h (FIG. 9B) and 24 h (FIG. 9C) with an increase of the fluorescence intensity at 24 h (FIG. 9C). Similar results were obtained with RAW 264.7 cells, as shown in FIGS. 15A-15C. These results support the hypothesized energy-dependent uptake mechanism of hGM-PMMA28 particles. Such an energy dependent uptake confirmes an active cellular uptake of hGM-PMMA28 particles by trans-membranal receptors and/or transporters.

A complementary assay was conducted by imaging flow cytometry (Amnis ImageStream®X Mark II-Digital, 4-laser imaging flow cytometer, Merck KGaA, Darmstadt, Germany). For each experiment, Rh 30 cells were incubated with nanoparticles and finally trypsinized and suspended in 50 μL of buffer (cold PBS) in 0.6 ml microcentrifuge tubes. Before running the samples, the ImageStream was calibrated using speedbeads. Samples were acquired in the order of untreated and treated with particles. In each experiment 5,000 events for each sample were acquired in the imaging flow cytometer equipped with the 405, 488, 560 and 642 nm lasers. Cells were acquired at 40× magnification. The INSPIRE® Acquisition Software was used for data collection. IDEAS® Analysis Software—used for the quantitative cellular image analysis and population statistics.

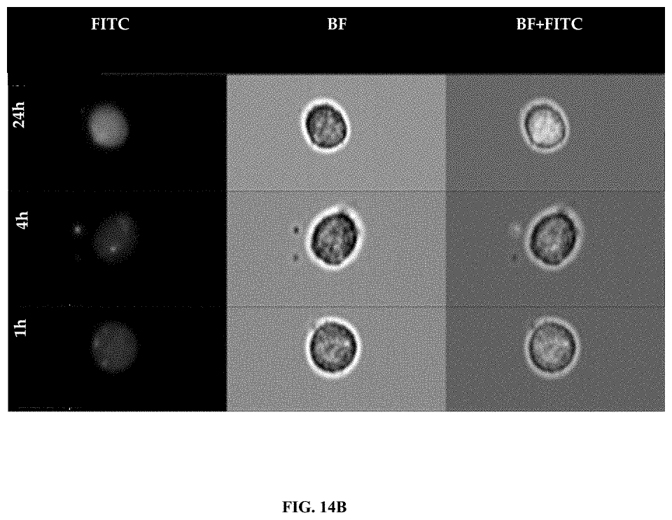

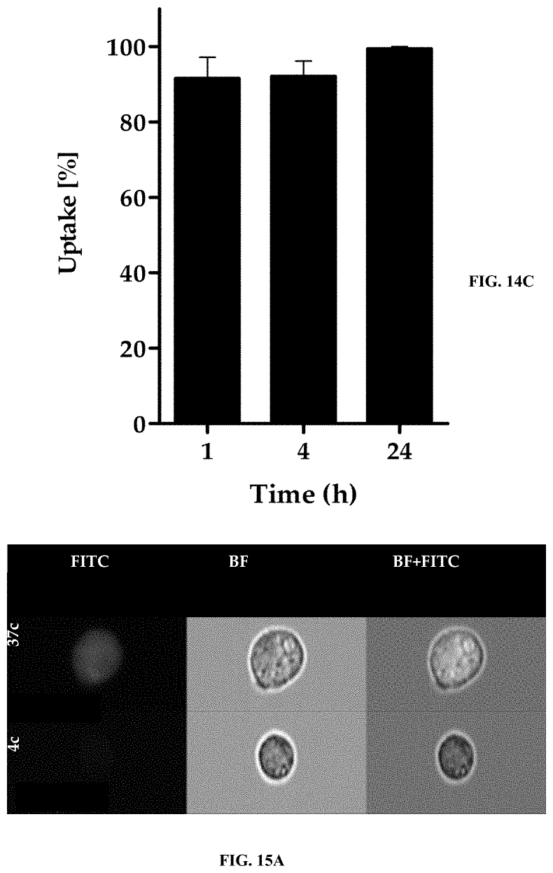

hGM-PMMA28 particles were rapidly internalized into RAW 264.7 cells (FIGS. 14A-14C), exhibiting an 80% uptake after 1 h incubation at 37° C.

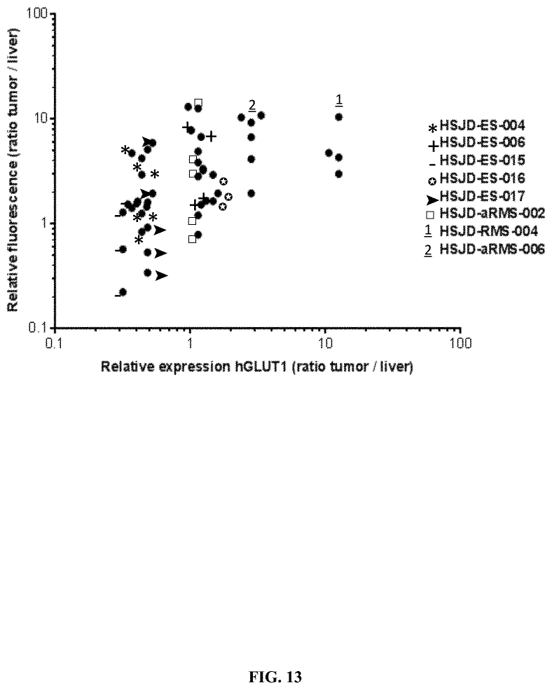

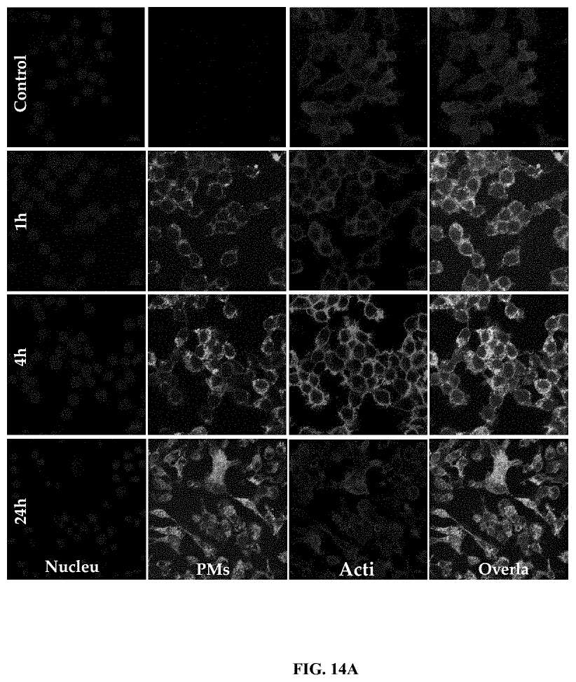

As shown in FIG. 13, the uptake of hGM-PMMA28 particles is in close correlation with a relative in-vivo expression of hGLUT-1 in various sarcoma tissues. These results further confirm the hGLUT-1 related cellular uptake mechanism of hGM-PMMA28 particles.

### Example 7

### Encapsulation of Imatinib in hGM-PMMA28 Particles

The encapsulation capacity of amphiphilic hGM-PMMA28 particles was studied with the tyrosine kinase inhibitor imatinib. For this, 5% w/v solution of hGM-PMMA28 in water was prepared and 1.7 mg imatinib per mL of particle suspension added and suspension was magnetically stirred at 25° C. (72 h). Then, the solution was freeze-dried, re-dispersed in methanol and the absorbance measured in microplate spectrophotometer (λ=272 nm). Then, the imatinib concentration was calculated by interpolation in a calibration curve in the range of 0.0004-0.01% w/v (R²=0.996). The same procedure was carried out without drug and with polymer and used as blank. The encapsulation efficiency using this methodology and polymer:imatinib weight ratio was 100%. The total drug loading is 3.4% w/w.

### Example 8

### Microscopy Visualization of hGM-PMMA28 Particles

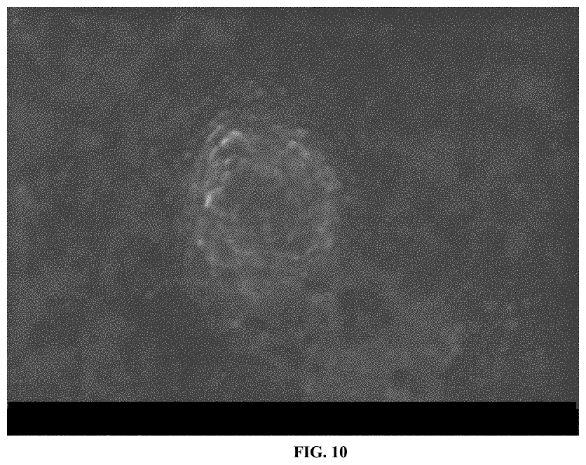

The morphology of imatinib-free hGM-PMMA28 particles was analyzed by high-resolution scanning electron microscopy (HRSEM, Zeiss Ultra-Plus FEG 0.02-30 kV SEM, Cambridge, Mass., USA). HRSEM samples of 1-5% w/v particles were prepared by drop casting on silicon wafer and carbon coated. The size of the imatinib-free particles was in the 160-180 nm range (FIG. 10).

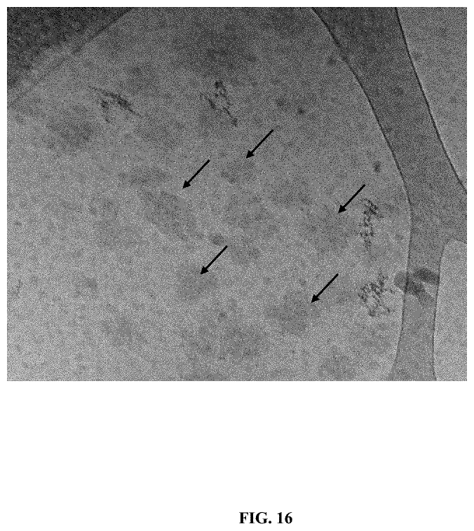

A dispersion of hGM-PMMA28 particles was analyzed by Cryo-TEM, and the results are presented in FIG. 16.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition-of-matter comprising one or more amphiphilic copolymers, said one or more amphiphilic copolymers comprising hydrophobic domain and a hydrophilic domain, wherein:
(a) each of said hydrophobic domain and said hydrophilic domain comprises a polymeric backbone having at least two monomeric units;
(b) the polymeric backbone of said hydrophobic domain is attached to the polymeric backbone of said hydrophilic domain; and
(c) said one or more amphiphilic copolymers are configured to form a multi-micellar structure, at a critical micellar concentration (CMC) of below than 4% w/v.

2. The composition-of-matter of claim 1, wherein at least a portion of said hydrophilic domain is capable of binding to a cell or a cell organelle via a carbohydrate cell receptor and/or transporter expressed on the outer surface of said cell or a cell organelle.

3. The composition-of-matter of claim 1, being in the form of multi-micellar structure, wherein said multi-micellar structure comprises a plurality of micelles having a size ranging from 1 nm to 10 μm.

4. The composition-of-matter of claim 1, wherein said hydrophobic domain comprises one or more monomeric units derived from a polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, and polyorthoester, or any combination thereof.

5. The composition-of-matter of claim 1, wherein said hydrophobic domain comprises one or more monomeric units derived from a lipidic molecule, a lipid or phospholipid selected from the group consisting of: fatty acid, fatty alcohol, or any combination thereof.

6. The composition-of-matter of claim 1, wherein said hydrophobic domain comprises one or more monomeric units derived from a polymer selected from the group consisting of: N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valerolactone, or any combination thereof.

7. The composition-of-matter of claim 1, wherein said hydrophilic domain comprises one or more monomeric units derived from a polymer selected from the group consisting of: alginate, galactomannan, hydrolyzed galactomannan, glucomannan, hydrolyzed glucomannan, guar gum, xanthan gum, pectin, hyaluronic acid, chondroitin sulfate, dermatan sulfate, keratin sulfate, levan heparin sulfate, beta-glucan, fucoidan, mannan, fucomannan, galactofucan, and fucan, or any combination thereof.

8. The composition-of-matter of claim 1, wherein the hydrophobic domain is present in a concentration of 2% to 90%, by weight, of the amphiphilic copolymer.

9. The composition-of-matter of claim 1, wherein said one or more amphiphilic copolymers are characterized as being substantially non-biodegradable for a period of at least 24 hours in a physiologic environment.

10. The composition-of-matter of claim 1, wherein said one or more amphiphilic copolymers are characterized as being biodegradable in a physiologic environment.

11. The composition-of-matter of claim 1, wherein said one or more amphiphilic copolymers are characterized by a hydrophilic-lipophilic balance (HLB) value that ranges from 1 to 24.

12. The composition-of-matter of claim 1, comprising one or more active agents, each of said active agents being independently encapsulated within or attached to said hydrophobic domain.

13. The composition-of-matter of claim 12, wherein said one or more active agents are characterized as being stably encapsulated within or attached to said hydrophobic domain in a physiological environment for at least 24 h.

14. The composition-of-matter of claim 12, wherein said one or more active agents are selected from the group consisting of a pharmaceutically active agent, a labeling agent, a diagnostic agent, a prophylactic agent, a surface-modifying agent, a tumor-targeting-ligand or moiety.

15. The composition-of-matter of claim 12, wherein said one or more active agents are water-insoluble agents.

16. The composition-of-matter of claim 1, wherein at least a portion of said hydrophilic domain of the multi-micellar structure is positively or negatively charged.

17. (canceled)

18. A pharmaceutical composition, comprising the composition-of-matter of claim 1 and a pharmaceutically acceptable carrier.

19. (canceled)

20. (canceled)

21. A method for treating a medical condition, comprising administering the pharmaceutical composition of claim 18 to a subject in need thereof, thereby treating said medical condition.

22. The method of claim 21, wherein said administering is affected orally, nasally, ocularly or by inhalation.

23. A process of preparing an amphiphilic copolymer being in the form of a self-assembled or a multi-micellar structure, the self-assembled or a multi-micellar structure further comprising one or more active agents, the process comprising the steps of:
grafting a hydrophobic polymeric backbone to a hydrophilic polymeric backbone, thereby forming an amphiphilic copolymer configured to form the self-assembled or a multi-micellar structure;
mixing the amphiphilic copolymer with a solvent at a concentration above a predefined minimal concentration thereby forming a dispersion, optionally wherein said predefined minimal concentration is a critical micellar concentration (CMC);
optionally, heating said amphiphilic copolymer in an aqueous medium to a temperature ranging from about 30° C. to about 50° C.; and
adding said one or more active agents to the dispersion, thereby forming an amphiphilic copolymer being in the form of the self-assembled or multi-micellar structure having attached thereto said one or more active agents.

24. (canceled)

25. (canceled)
